(19) United States
(12) Patent Application Publication
(10) Pub. No.: US 2022/0009891 A1
(43) Pub. Date: Jan. 13, 2022

(54) (R) ENANATIOMER OF APLEXONE

(71) Applicant: The Regents of the University of California, Oakland, CA (US)

(72) Inventors: Oyhun KWON, Los Angeles, CA (US); Qihai XU, Los Angeles, CA (US); Nathan J. DUPPER, Los Angeles, CA (US); Jau-Nian CHEN, Los Angeles, CA (US); Adam D. LANGENBACHER, Los Angeles, CA (US)

(21) Appl. No.: 17/278,230

(22) PCT Filed: Sep. 19, 2019
(86) PCT No.: PCT/US2019/051823

§ 371 Date: Mar. 19, 2021

Related U.S. Application Data
(60) Provisional application No. 62/734,081, filed on Sep. 20, 2018.

(51) Int. Cl. C07D 211/96; A61K 45/06

(52) U.S. Cl. CPC C07D 211/96; A61K 45/06

(57) ABSTRACT

R-aplexone, methods of preparing R-aplexone, or aplexone substantially free of its S-stereoisomer are disclosed. Methods of using R-aplexone or a pharmaceutically acceptable salt or solvate thereof to treat and/or prevent disease are disclosed. Methods of preparing 6-substituted tetrahydropyridine motifs with high enantiomeric excess are also disclosed.

### CROSS-REFERENCE TO RELATED APPLICATIONS

This application claims the benefit of priority of U.S. Provisional Patent Application No. 62/734,081 filed Sep. 20, 2018. The entire contents of the above-referenced disclosure is specifically incorporated herein by reference without disclaimer.

### STATEMENT REGARDING FEDERALLY FUNDED RESEARCH

This invention was made with government support under Grant Numbers GM071779 and HL129052, awarded by the National Institutes of Health. The government has certain rights in the invention.

### BACKGROUND

Functionalized tetrahydropyridines and piperidines are common structural motifs in many biologically active natural products (Michael, J. P. Nat. Prod. Rep. 2004, 21, 625; Michael, J. P. Simple indolizidine alkaloids. The Alkaloids; Cordell, G. A., Ed.; Academic Press: San Diego, 2001; Vol. 55; Dewick, P. M. Medicinal Natural Products; John Wiley and Sons: Chichester, 1997, Chapter 6; Pinder, A. R. Nat. Prod. Rep. 1992, 9, 491) and synthetic pharmaceuticals (Rubiralta, M. et al. Piperidine. Structure, Preparation, Reactivity and Synthetic Applications of Piperidine and Its Derivatives; Elsevier: Amsterdam, 1991). In fact, piperidine is the most frequently encountered heterocycle in the top-200 drug list (Vitaku et al. J. Med. Chem. 2014, 57, 10257; McGrath, et. al. J. Chem. Educ. 2010, 87, 1348). One particular piperidine structure, the guvacine moiety having the following structure:

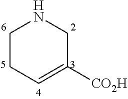

has been identified as the pharmacophore of the γ-aminobutyric acid (GABA) uptake inhibitor (Mateeva et al., Curr. Med. Chem. 2005, 12, 551; Felpin et al., Curr. Org. Synth. 2004, 1, 83; N'Goka et al., J. Med. Chem. 1991, 34, 2547; Czapinski et al., Curr. Top. Med. Chem. 2005, 5, 3 and references cited therein; Dalby, Eur. J. Pharmacol. 2003, 479, 127; Bohme et al., Curr. Med. Chem. 2001, 8, 1257; Krogsgaard-Larsen et al., Curr. Pharm. Des. 2000, 6, 1193; GABA in Nervous System Function, Roberts et al., Eds., Raven Press: New York, 1976; Yunger, et al., J. Pharmacol. Exp. Ther. 1984, 228, 109; Johnston et al., Systematic study of GABA analogues of restricted conformation, GABA-Neurotransmitters: Pharmacochemical, Biochemical and Pharmacological Aspects, Krogsgaard-Larsen, et al., Eds., Munks-gaard: Copenhagen, Denmark, 1978, p 147; Johnston et al., J. Neurochem. 1976, 26, 1029; Bisel et al., Bioorg. Med. Chem. Lett. 1996, 6, 3025). GABA is one of the major mammalian inhibitory neurotransmitters, and a number of diseases, including Parkinson's, Huntington's, epilepsy, and schizo-phrenia, have been linked to the dysfunction of GABAergic synapses (Czapinski et al., 2005; Dalby, 2003; Bohme et al., 2001; Krogsgaard-Larsen et al., 2000; GABA in Nervous System Function, 1976). Modification at the C6 position of guvacine, as in the following GABA uptake inhibitor compound structure:

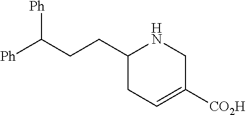 [SMILES: O=C(O)C1=CCC(CCC(c2ccccc2)c2ccccc2)NC1]

can also result in good GABA uptake inhibition (N'Goka et al., 1991; Bisel et al., 1996). Recently, another guvacine derivative, aplexone, having the structure of Formula I:

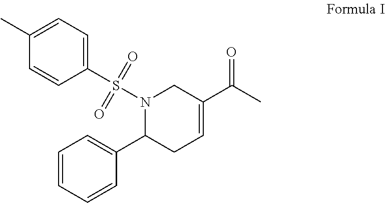 [SMILES: CC(=O)C1=CCC(c2ccccc2)N(S(=O)(=O)c2ccc(C)cc2)C1]

was shown to regulate arteriovenous angiogenesis in zebrafish by acting on the HMG-CoA reductase pathway (Choi, et al., Development 2011, 138, 1173). In the same zebrafish studies, it was found that aplexone lowered the levels of embryonic cholesterol to a degree similar to that of atorvastatin (trade name: Lipitor). Many diseases or conditions exist which can be benefited by a reduction in angiogenesis or a lowering of cell cholesterol levels.

A survey of the literature revealed a limited number of general methods for the enantioselective synthesis of 6-substituted guvacines (Ramachandran, et al., J. Org. Chem. 2005, 70, 7911; Clinch, et al., Tetrahedron 1989, 45, 239; Langlois, et al., Tetrahedron 1975, 31, 419). Ramachandran's group reported the preparation of C6-chiral guvacine derivatives. They installed the C6-chiral center through the allylation of imines using (−)-B-allyldiisopinocampheylborane, in lengthy syntheses (Ramachandran, et al., 2005), but enantiomeric excess (ee) greater than 90% was not achieved:

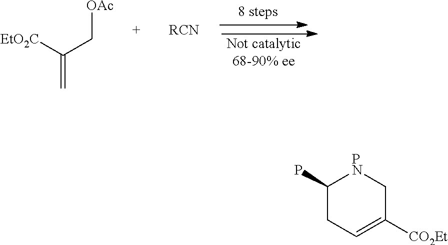

Previously, a general method was reported for the synthesis of functionalized tetrahydropyridines through phosphine-catalyzed [4+2] annulation of imines with 2-alkyl-2,3-butadienoates (Zhu, et al., J. Am. Chem. Soc. 2003, 125, 4716; Lu, et al., Org. Synth. 2009, 86, 212; Soriano, et al., Chem. Soc. Rev. 2014, 43, 3041; Lopez, et al., Chem. Soc. Rev. 2014, 43, 2904; Wang, et al., Chem. Soc. Rev. 2014, 43, 2927; Yu, et al., Angew. Chem., Int. Ed. 2012, 51, 3074; Krause, et al., Modern Allene Chemistry; Wiley-VCH: Weinheim, 2004; Taylor, Chem. Rev. 1967, 67, 317). A catalytic asymmetric version of this transformation, using a chiral phosphine catalyst, was presented by Fu; while high enantiomeric excess values (“ee”) were obtained in the syntheses of 2,6-disubstituted guvacines, 6-substituted guvacines were formed with only moderate enantioselectivities (Wurz, et al., J. Am. Chem. Soc. 2005, 127, 12234). Zhao and colleagues also developed an enantioselective synthesis of 2,6-disubstituted guvacines catalyzed by an amino acid-derived bifunctional phosphine; under their conditions, however, the 6-phenyl guvacine ester could not be obtained (Xiao, et al., Chem.-Eur. J. 2011, 17, 10562):

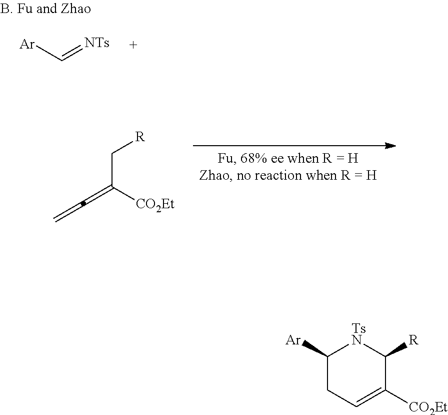

Furthermore, Guo and Sasai both reported catalytic asymmetric [4+2] annulations of sulfamate-derived cyclic imines with allenoates; again, they obtained high ee's only for 2,6- or 6,6-disubstituted guvacine derivatives, respectively (Yu, et al., Tetrahedron 2014, 70, 340; Takizawa, et al., Asian J. Org. Chem. 2014, 3, 412):

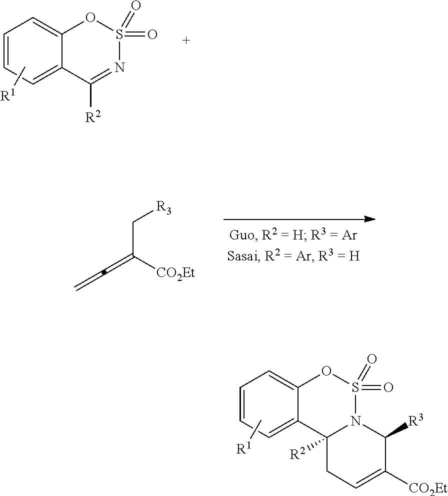

There remains a need for additional methods for synthesizing 6-substituted tetrahydropyridine motifs with high enantiomeric excess.

### SUMMARY

Herein is described a process for synthesis of 6-substituted tetrahydropyridine motifs with high enantiomeric excess. The methods described herein overcome the current limitations in preparing 6-substituted guvacine esters with high enantiomeric excess. The method is premised in part on a catalytic asymmetric [4+2] annulation of a simple α-methylallenoate.

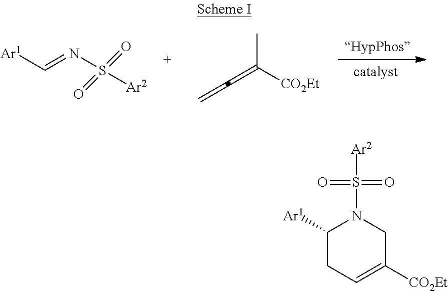

wherein Ar¹ and Ar² are independently an aryl.

Further, it is herein disclosed that the R-stereoisomer of aplexone, R-aplexone (Formula II), is surprisingly the active enantiomer, while the S-stereoisomer of aplexone, S-aplexone (Formula VIII) shows little to no biological activity in the biological assays used herein. Accordingly, lower concentrations R-aplexone or R-aplexone substantially free of S-aplexone are needed to treat a subject as compared to a racemic mixture of R and S-stereoisomers of aplexone. Further, any side effects that may be caused by S-aplexone can also be avoided without sacrificing efficacy when R-aplexone is used.

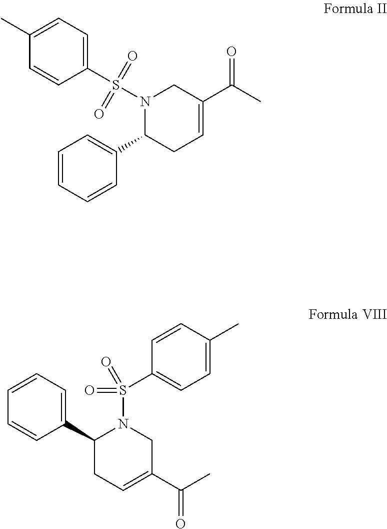 [SMILES: CC(=O)C1=CC[C@H](c2ccccc2)N(S(=O)(=O)c2ccc(C)cc2)C1 ; CC(=O)C1=CC[C@@H](c2ccccc2)N(S(=O)(=O)c2ccc(C)cc2)C1]

Certain embodiments of the current disclosure concern methods and compositions involving aplexone, having a formula of Formula I, 1-(6-phenyl-1-tosyl-1,2,5,6-tetrahydropyridin-3-yl)ethan-1-one, substantially free of its S-stereoisomer. A composition is “substantially free” of S-aplexone if it includes a mixture of R-aplexone and (optionally) S-aplexone wherein the weight of S-aplexone, if present, is no more than about 10% of the total weight of R-aplexone and S-aplexone in the composition. In some embodiments, the composition may contain no more than about 10, 9, 8, 7, 6, 5, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, or 1.0% or any range derivable therein by weight of S-aplexone relative to the total weight of R-aplexone and S-aplexone in the composition.

In certain embodiments, the ratio of R-aplexone to S-aplexone in a composition is greater than about 90:10 by weight, greater than about 95:5 by weight, greater than about 99:1 by weight, or any range therein. Certain embodiments are directed to R-aplexone.

Certain embodiments are directed to a pharmaceutically acceptable salt or solvate of aplexone, substantially free of its S-stereoisomer.

In some aspects, disclosed is a pharmaceutical composition comprising a pharmaceutically acceptable carrier and aplexone substantially free of its S-stereoisomer. In certain embodiments, the ratio of R-aplexone to S-aplexone in the pharmaceutical composition is greater than 90:10 by weight. In certain embodiments, the ratio of R-aplexone to S-aplexone in the pharmaceutical composition is greater than 95:5 by weight. In certain embodiments, the ratio of R-aplexone to S-aplexone in the pharmaceutical composition is greater than 98:2 by weight. In certain embodiments, the ratio of R-aplexone to S-aplexone in the pharmaceutical composition is greater than 99:1 by weight. In certain embodiments, the ratio of R-aplexone to S-aplexone in the pharmaceutical composition is greater than 99.5:0.5 by weight. In certain further embodiments, the pharmaceutical composition, further comprises one or more additional pharmaceutically active agent(s). In some instances, the additional pharmaceutically active agent(s) is effective for lowering cellular cholesterol levels and/or for cancer treatment.

In some aspects, disclosed is a method for treatment or prevention of a disease or condition comprising administration of the compound and/or compositions disclosed herein. Certain embodiments are directed to a method for lowering cholesterol levels in a subject comprising administering to the subject an therapeutically effective amount of the compound and/or compositions disclosed herein. In some instances, the administration of the compound and/or compositions disclosed herein lowers cellular cholesterol levels in the subject. Certain embodiments are directed to a method for inhibiting angiogenesis in a subject comprising administering to the subject an therapeutically effective amount of the compound and/or compositions disclosed herein. Certain embodiments are directed to a method for treating angiogenesis-mediated condition or disease in a subject comprising administering to the subject an therapeutically effective amount of the compound and/or compositions disclosed herein. In certain embodiments, the angiogenesis-mediated condition or disease is an inflammatory disease. In certain embodiments, the angiogenesis-mediated condition or disease is a cancer. Certain embodiments are directed to a method for reducing amount of 3-hydroxy-3-methylglutaryl-coenzyme A reductase comprising administering to the subject an therapeutically effective amount of the compound and/or compositions disclosed herein.

In certain embodiments, the composition is greater than 10% more effective at treating the subject as compared to treating with the same amount by weight of a mixture of aplexone wherein R-aplexone is not in enantiomeric excess. In certain embodiments, the composition is greater than 20% more effective at treating the subject as compared to treating with the same amount by weight of a mixture of aplexone wherein R-aplexone is not in enantiomeric excess. In certain embodiments, the composition is greater than 30% more effective at treating the subject as compared to treating with the same amount by weight of a mixture of aplexone wherein R-aplexone is not in enantiomeric excess. In certain embodiments, the composition is greater than 40% more effective at treating the subject as compared to treating with the same amount by weight of a mixture of aplexone wherein R-aplexone is not in enantiomeric excess. In certain embodiments, the composition is greater than 50% more effective at treating the subject as compared to treating with the same amount by weight of a mixture of aplexone wherein R-aplexone is not in enantiomeric excess.

In certain further embodiments, R-aplexone is used in combination with another pharmaceutically active agent, such as a cholesterol lowering agent or an agent effective for cancer treatment. In certain embodiments, the subject is human.

Certain embodiments are directed to methods of preparing a compound having a general formula of Formula III and with enantiomeric excess greater than about 80%, comprising reacting imine, a compound having a general formula of Formula IV with a compound with general formula V according to scheme I, wherein the reaction is catalyzed by HypPhos catalyst. In certain embodiments a compound with general formula of Formula III is prepared according to scheme I with enantiomeric excess greater than about 90%. In certain embodiments a compound with general formula of Formula III is prepared according to scheme I with enantiomeric excess greater than about 95%. In certain embodiments a compound with general formula of Formula III is prepared according to scheme I with enantiomeric excess greater than about 98%. In certain embodiments a compound with general formula of Formula III is prepared according to scheme I with enantiomeric excess about 99% or higher. In certain embodiments, the HypPhos catalyst is a compound having a general formula of formula VI.

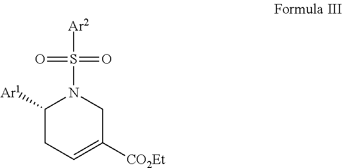

wherein Ar¹ and Ar² are independently an aryl.

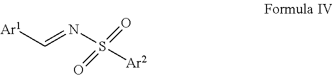

wherein Ar¹ and Ar² are independently an aryl.

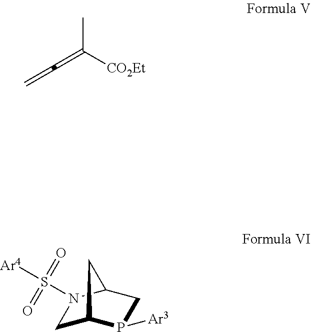

wherein Ar³ and Ar⁴ are independently an aryl, and the configuration at the phosphorus stereocenter is R.

In certain embodiments, the compound of general formula III has the structure of Formula VII and ratio R-stereoisomer to S-stereoisomer of the compound of formula VII obtained from the reaction of scheme I is greater than 90:10 by weight.

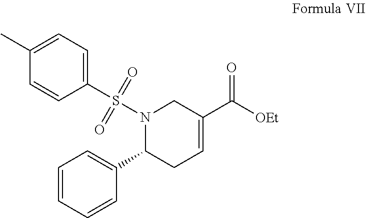

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention.

It is also specifically contemplated that, in certain embodiments, one or more compounds whose structure is covered by Formula III, IV, VI or VIII may be excluded.

Throughout this application, the term “about” is used to indicate that a value includes the inherent variation of error for the device, the method being employed to determine the value, or the variation that exists among the study subjects.

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and also covers other unlisted steps.

As used herein, the term “IC50” refers to an inhibitory dose that results in 50% of the maximum response obtained.

The term half maximal effective concentration (EC50) refers to the concentration of a drug that presents a response halfway between the baseline and maximum after some specified exposure time.

The terms “inhibiting”, “reducing,” or “prevention,” or any variation of these terms, when used in the claims and/or the specification includes any measurable decrease or complete inhibition to achieve a desired result.

The use of the term “or” in the claims is used to mean “and/or” unless explicitly indicated to refer to alternatives only or the alternatives are mutually exclusive, although the disclosure supports a definition that refers to only alternatives and “and/or.”

As used herein, the term “patient” or “subject” refers to a living mammalian organism, such as a human, monkey, cow, sheep, goat, dogs, cat, mouse, rat, guinea pig, or species thereof. In certain embodiments, the patient or subject is a primate. Non-limiting examples of human subjects are adults, juveniles, infants and fetuses.

The use of the word “a” or “an” when used in conjunction with the term “comprising” in the claims and/or the specification may mean “one,” but it is also consistent with the meaning of “one or more,” “at least one,” and “one or more than one.”

“Effective amount” and “therapeutically effective amount” are used interchangeably herein, and refer to an amount of a composition or a compound or a pharmaceutically acceptable salt or solvate thereof, as described herein, effective to achieve a particular biological or therapeutic result such as, but not limited to, the biological or therapeutic results disclosed herein. A therapeutically effective amount of the composition or a compound or a pharmaceutically acceptable salt or solvate thereof may vary according to factors such as the disease state, age, sex, and weight of the individual, and the ability of the antibody or functional fragment thereof to elicit a desired response in the individual. Such results may include, but are not limited to, suppression of angiogenesis, particularly venous angiogenesis, reduction of cholesterol in a cell, and/or treatment of angiogenesis-mediated condition or disease, as determined by any means suitable in the art.

Other objects, features and advantages of the present invention will become apparent from the following detailed description. It should be understood, however, that the detailed description and the specific examples, while indicating embodiments of the invention, are given by way of illustration only, since various changes and modifications within the spirit and scope of the invention will become apparent to those skilled in the art from this detailed description.

It is contemplated that embodiments described in the context of the term “comprising” may also be implemented in the context of the term “consisting of” or “consisting essentially of.”

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Use of the one or more compositions may be employed based on methods described herein. Use of one or more compositions may be employed in the preparation of medicaments for treatments according to the methods described herein. Other embodiments are discussed throughout this application. The embodiments in the Example section are understood to be embodiments that are applicable to all aspects of the technology described herein.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Relative cellular levels of cholesterol in zebrafish embryos treated with a mixture of aplexone enantiomers ((±)-AP), (R) aplexone enantiomer ((R)-AP), (S) aplexone enantiomer ((S)-AP), and atorvastatin (AT), all at 40 μM; DMSO used as a control.

FIG. 2. Caudal vein regions of two-day-old zebrafish embryos treated with various concentrations of aplexone (3). Formation of the caudal vein plexus is disrupted by treatment with 10 μM or higher of (±)-aplexone (3) and 5 μM or higher of (R)-aplexone (3), but the caudal vein plexus forms normally even after treatment with 20 μM (S)-aplexone (3).

FIG. 3. Supercritical fluid chromatography (SFC) and HPLC traces of 7c (Formula VII, R-isomer).

FIG. 4. SFC and HPLC traces of R-aplexone (Formula II).

FIG. 5. SFC and HPLC traces of ent-7c (S-isomer of Formula VII).

FIG. 6. SFC and HPLC traces of S-aplexone (S-isomer of Formula I).

FIGS. 7A and 7B. Structures of compounds of Formula III.

FIG. 8. Structures of compounds of Formula IV.

FIG. 9. Structures of compounds of Formula VI that can be used in methods to produce 6-substituted tetrahydropyridine motifs with enantiomeric excess.

### DETAILED DESCRIPTION

Previous results, disclosed in U.S. Pat. No. 9,084,782, which is incorporated by reference herein, from the inventors have identified aplexone and functional variants thereof target the HMG-CoA reductase (HMGCR) pathway (e.g., elevate transcription of enzymes in the HMGCR pathway, which results in a decreased amount of HMGCR protein); effectively suppress angiogenesis, particularly venous angiogenesis (rather than arterial angiogenesis); inhibit prenylation (both famesylation and geranlygeranylation) of cellular proteins (e.g., ras and rhoA); block cell proliferation and migration; and reduce cellular cholesterol levels. Results of the present disclosure show R-aplexone is the active enantiomer. Further, methods of making R-aplexone and other enantiomericly enriched molecules are disclosed herein.

### Phosphine-Catalyzed [4+2] Allenoate-Imine Annulation

HypPhos catalyzed, [4+2] annulation between an imine and an allenoate can be performed according to scheme I.

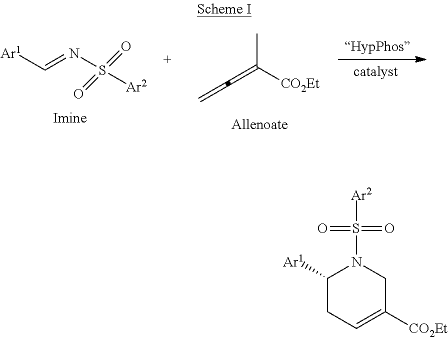

wherein Ar¹ and Ar² is independently an aryl.

In certain embodiments, the HypPhos is a compound with the general formula of formula VI. In certain embodiments, Ar³ is phenyl, p-flurophenyl, p-anisyl, 1-naphthyl or 2-naphthyl and Ar⁴ is p-MeC₆H₄ and the configuration at the phosphorus stereocenter is R. In certain embodiment, an additive is added to prevent hydrolysis of Imines. In certain embodiments, the additive is 4 Å molecular sieves or acetic acid. In certain embodiment, acetone, benzene, tetrahydrofuran, dichloromethane or a mixture thereof is used as solvent in the reaction. In certain embodiments dichloromethane is used as solvent in the reaction. In certain embodiments the reaction is performed at ambient temperature and pressure. In certain embodiments the reaction of performed for 10 to 72 hours. In certain embodiments Ar¹ is phenyl, o-MeOC₆H₄, m-MeOC₆H₄, p-MeOC₆H₄, p-MeC₆H₄, o-ClC₆H₄, m-ClC₆H₄, p-ClC₆H₄, p-BrC₆H₄, p-FC₆H₄, p-NCC₆H₄, p-NCC₆H₄, 2-furyl, 2-thienyl or 2-N-methylpyrrolyl and Ar² is phenyl, p-nitroC₆H₄, p-MeC₆H₄, p-ClC₆H, p-BrC₆H or o-nitroC₆H₄. In certain embodiments the reaction is performed according to example I.

### Synthesis of R-aplexone

Aplexone or aplexone variants can be synthesized by treating corresponding compounds having general formula of Formula III with Tebbe reagent. R-aplexone can be synthesized according to scheme II. In certain embodiments R-aplexone is synthesized according to example II.

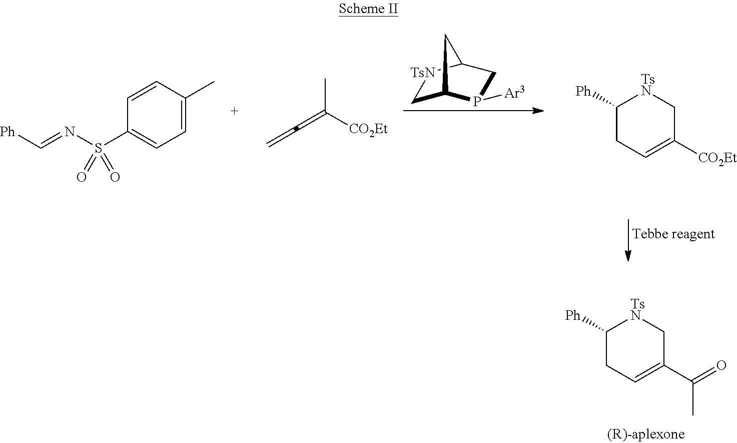

wherein Ar³ is phenyl, p-fluorophenyl, p-anisyl, 1-naphthyl or 2-naphthyl.

Enantiomeric excess (“ee”) represents the percentage by which one enantiomer, such as a (R) or (S) isomer, exceeds the other in a scalemic mixture, a mixture of the enantiomers at a ratio other than 1:1. Enantiomeric excess may be calculated as [((R−S)/(R+S))*100], wherein “R” and “S” are the moles of the (R) and (S) isomers, respectively, that are present in a composition or sample. High enantiomeric excess refers to ee greater than about 80, 81, 82, 83, 84, 85, 86, 87, 88, 89, 90, 91, 92, 93, 94, 95, 96, 97, 98% or 99% or any range derivable therein. In certain embodiments R-aplexone, with enantiomeric excess about 99% was synthesized according to scheme III

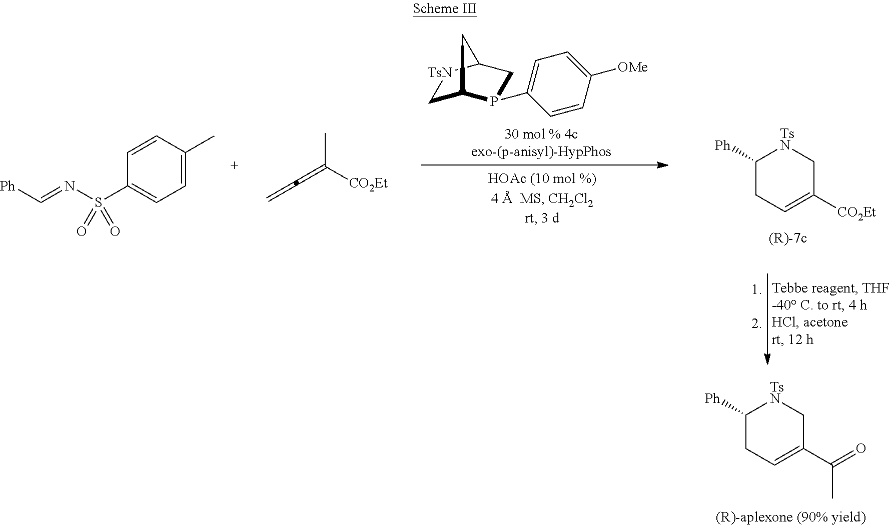

### Use of R-aplexone

As described herein, R-aplexone and functional variants thereof are effective for reducing cellular cholesterol levels and thus are useful for treating subjects having undesirably high levels of cholesterol (hypercholesterolemia). Further, as described herein, R-aplexone and functional variants thereof are effective for reducing angiogenesis and thus are useful for treating subjects having undesirably high levels of angiogenesis, such as angiogenesis associated with cancers.

Plasma cholesterol levels have been positively correlated with the incidence of clinical events associated with coronary heart disease (CHD). Thus, pharmacological interventions that reduce cholesterol levels in mammals have a beneficial effect on CHD. In particular, decreased plasma levels of cholesterol (e.g., low density lipoprotein (LDL) cholesterol) levels are associated with decreased atherosclerosis and a decreased risk of CHD, and hypolipidemic agents used in either monotherapy or combination therapy are effective at reducing plasma LDL cholesterol levels and the subsequent risk of CHD.

Cholesterol synthesis occurs in multiple tissues, but principally in the liver and the intestine. It is a multistep process starting from acetyl-coenzyme A catalyzed by a series of enzymes including HMG-CoA reductase, HMG-CoA synthase, squalene synthetase, squalene epoxidase, squalene cyclase and lanosterol demethylase. Without wishing to be bound by any particular mechanism, it is suggested that inhibition of catalysis by these enzymes or blocking HMG-CoA reductase gene expression may be an effective means to reduce cholesterol biosynthesis, and can lead to a reduction in cholesterol levels. For example, there are known HMG-CoA reductase inhibitors (e.g., lovastatin, simvastatin, pravastatin, fluvastatin, atorvastatin, rivastatin, cerivastatin, nisvastatin) that are used for the treatment of hypercholesterolemia.

“Angiogenesis,” as used here, refers to the development of new blood vessels, generally capillaries, from pre-existing vasculature. Venous angiogenesis originates from veins; arterial angiogenesis originates from arteries. Normal angiogenesis is activated during tissue growth, from embryonic development through maturity, and then enters a period of relative quiescence during adulthood. Normal angiogenesis is also activated during wound healing, and at certain stages of the female reproductive cycle. Inappropriate (aberrant) angiogenesis (e.g., at greater levels than in “normal” subjects) has been associated with a number of conditions or disease states. Such conditions or diseases are referred to herein as “angiogenesis-mediated” conditions or diseases. An “angiogenesis mediated” condition or disease is a condition or disease which is, at least in part, caused by aberrant or inappropriate angiogenesis, or in which symptoms result from, at least in part, aberrant or inappropriate angiogenesis. Various forms of inflammatory diseases, including chronic inflammatory disorders, can be treated by a method of the current disclosure.

In cancer, the growth of solid tumors has been shown to be angiogenesis-dependent; and the inhibition of angiogenesis has been reported to reduce tumor growth, particularly wherein the cancer is characterized by cancer cells that have not yet been vascularized to form a solid tumor. Among the types of cancer that can be treated by a method of the of the current disclosure are, e.g., basal cell carcinoma or other solid tumors, medulloblastoma, small cell lung cancer, pancreatic cancer, stomach cancer, esophageal cancer, colorectal cancer, ovarian cancer, multiple myeloma, leukemia, prostate cancer and breast cancer.

Among the conditions or diseases that can be treated by a method of the invention are, e.g., cancer, rheumatoid arthritis osteoarthritis, asthma, pulmonary fibrosis, various retinopathies, including diabetic retinopathy, macular degeneration, angiofibroma, neovascular glaucoma, arteriovenous malformation, nonunion fracture, connective tissue disorder, spider veins, Osler-Weber syndrome, atherosclerotic plaque, psoriasis, corneal graft neovascularization, pyogenic granuloma, retrolental fibroplasia, scleroderma, granulations, hemangioma (e.g. in infants), trachoma, hemophilic joints, vascular adhesions, angiofibroma of the nasopharynx, avascular necrosis of bone, endometriosis, metastasis, ischemic disease, atherosclerosis, acute coronary syndromes, stroke, and peripheral vascular diseases (e.g. peripheral ischemia).

### Diseases to be Treated or Prevented

Some embodiments of the present invention concern methods of treating a patient. The patient may have any disease or condition for which treatment of R-aplexone is indicated. Examples of such diseases and conditions are discussed elsewhere in this specification.

“Treatment” and “treating” as used herein refer to administration or application of a therapeutic agent to a subject or performance of a procedure or modality on a subject for the purpose of obtaining a therapeutic benefit of a disease or health-related condition. For example, a pharmaceutical composition that includes R-aplexone may be administered to a subject to inhibit angiogenesis, or lower cellular cholesterol levels.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease.

### Routes of Administration

Administration of the pharmaceutical compositions comprising R-aplexone set forth herein may be by any number of routes including, but not limited to oral, intravenous, intramuscular, intra-arterial, intramedullary, intrathecal, intraventricular, intradermal, intratracheal, intravesicle, intraocular, transdermal, subcutaneous, intraperitoneal, intranasal, enteral, topical, sublingual, or rectal. Further details on techniques for formulation and administration may be found in the latest edition of Remington's Pharmaceutical Sciences (Maack Publishing Co., Easton, Pa.). In certain embodiments, R-aplexone is formulated for oral administration.

### Formulations

Where clinical applications are contemplated, pharmaceutical compositions will be prepared in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Aqueous compositions of the present invention can comprise an effective amount of the vector or compound(s), dissolved or dispersed in a pharmaceutically acceptable carrier or aqueous medium. The phrase “pharmaceutically” or “pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present invention, its use in therapeutic compositions is contemplated. Supplementary active ingredients also can be incorporated into the compositions, provided they do not inactivate the other active ingredients of the compositions.

The active compositions of the present invention can include classic pharmaceutical preparations. Administration of these compositions according to the present invention may be via any common route so long as the target tissue is available via that route. This includes oral, nasal, or buccal. Alternatively, administration may be by intradermal, subcutaneous, intramuscular, intraperitoneal or intravenous injection, or by direct injection into cardiac tissue. Such compositions would normally be administered as pharmaceutically acceptable compositions, as described supra.

The active compounds can also be administered parenterally or intraperitoneally. By way of illustration, solutions of the active compounds as free-base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations generally contain a preservative to prevent the growth of microorganisms.

The pharmaceutical forms suitable for injectable use can include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For oral administration the compounds of the present invention generally may be incorporated with excipients and used in the form of ingestible tablet, pill, capsule, etc.

The compositions of the present invention generally may be formulated in a neutral or salt form. Pharmaceutically-acceptable salts include, for example, acid addition salts derived from inorganic acids (e.g., hydrochloric or phosphoric acids), or from organic acids (e.g., acetic, oxalic, tartaric, mandelic, and the like). Salts formed with can also be derived from inorganic bases (e.g., sodium, potassium, ammonium, calcium, or ferric hydroxides) or from organic bases (e.g., isopropylamine, trimethylamine, histidine, procaine and the like).

Upon formulation, solutions can be administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Sterile aqueous media can be employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### Controlled/Extended/Sustained/Prolonged Release Administration

Another aspect of this invention provides methods of treating patients by delivering the pharmaceutical compositions set forth herein as a controlled release formulation. As used herein, the terms “controlled,” “extended,” “sustained,” or “prolonged” release of the composition of the present invention will collectively be referred to herein as “controlled release,” and includes continuous or discontinuous, and linear or non-linear release of the composition of the present invention. There are many advantages for a controlled release formulation of R-aplexone.

a. Tablets

A non-limiting controlled release tablet suitable for purposes of this invention is disclosed in U.S. Pat. No. 5,126,145, which is incorporated by reference herein. This tablet comprises, in admixture, about 5-30% high viscosity hydroxypropyl methyl cellulose, about 2-15% of a water-soluble pharmaceutical binder, about 2-20% of a hydrophobic component such as a waxy material, e.g., a fatty acid, and about 30-90% active ingredient.

### Medical Devices

Another embodiment contemplates the incorporation of R-aplexone or a composition comprising R-aplexone as set forth herein into a medical device that is then positioned to a desired target location within the body, whereupon the R-aplexone elutes from the medical device. As used herein, “medical device” refers to a device that is introduced temporarily or permanently into a mammal for the prophylaxis or therapy of a medical condition. These devices include any that are introduced subcutaneously, percutaneously or surgically to rest within an organ, tissue or lumen. Medical devices include, but are not limited to, stents, synthetic grafts, artificial heart valves, artificial hearts and fixtures to connect the prosthetic organ to the vascular circulation, venous valves, abdominal aortic aneurysm (AAA) grafts, inferior venal caval filters, catheters including permanent drug infusion catheters, embolic coils, embolic materials used in vascular embolization (e.g., PVA foams), mesh repair materials, a Dracon vascular particle orthopedic metallic plates, rods and screws and vascular sutures.

### Dosages

The amount of R-aplexone or composition comprising R-aplexone that is administered to a subject can be about, at least about, or at most about 0.1, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 600, 700, 800, 900, or 1000 mg of total R-aplexone, or any range derivable therein. Alternatively, the amount administered may be about, at least about, or at most about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 mg/kg of R-aplexone, or any range derivable therein, with respect to the weight of the subject. In certain embodiments, pharmaceutical compositions may comprise, for example, at least about 0.1% of an active compound. In other embodiments, the an active compound may comprise between about 2% to about 95% of the weight of the unit or between about 5% to about 75%, or between about 25% to about 60%, for example, and any range derivable therein.

The actual dosage amount of a composition of the present invention administered to a subject can be determined by physical and physiological factors such as body weight, severity of condition, the type of disease being treated, previous or concurrent therapeutic interventions, idiopathy of the patient and on the route of administration. The number of doses and the period of time over which the dose may be given may vary. The practitioner responsible for administration can, in any event, determine the concentration of active ingredient(s) in a composition and appropriate dose(s), as well as the length of time for administration for the individual subject. Typically, an effective dosage for the compound is in the range of about 0.01 mg/kg/day to 100 mg/kg/day in single or divided doses, preferably 0.1 mg/kg/day to 20 mg/kg/day in single or divided doses. Doses of about, at least about, or at most about 0.01, 0.05, 0.1, 0.15, 0.20, 0.25, 0.30, 0.35, 0.40, 0.45, 0.50, 0.55, 0.60, 0.65, 0.70, 0.75, 0.80, 0.85, 0.90. 0.95, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/kg/day, or any range derivable therein.

When provided in a discrete amount, each intake of R-aplexone or composition comprising R-aplexone can be considered a “dose.” A medical practitioner may prescribe or administer multiple doses over a particular time course (treatment regimen) or indefinitely.

The pharmaceutical composition may be administered 1, 2, 3, 4, 5, 6, 7, 8, 9, 10, 11, 12, 13, 14, 15, 16, 17, 18, 19, 20, 30, 40, 50, 60, 70, 80, or more times or any range derivable therein. It is further contemplated that R-aplexone may be taken for an indefinite period of time or for as long as the patient exhibits symptoms of the medical condition for which the therapeutic agent was prescribed. Also, R-aplexone may be administered every 2, 3, 4, 5, 6, 7, 8, 9, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, or 1, 2, 3, 4, 5, 6, 7 days, or 1, 2, 3, 4, 5 weeks, or 1, 2, 3, 4, 5, 6, 7, 8, 9, 10, 11, 12 months or more, or any range derivable therein. Alternatively, it may be administered systemically over any such period of time and be extended beyond more than a year.

### Other Therapeutic Options

In certain embodiments, it is envisioned to use R-aplexone in combination with other therapeutic modalities. For the treatment of a disease or condition characterized by high blood levels of cholesterol, R-aplexone can be combined or co-administered with a variety of suitable cholesterol-lowering agents, including, e.g., a variety of statins, such as, e.g., atorvastatin, marketed as Lipitor or Torvast (manufactured by Pfizer), fluvastatin (Lescol), lovastatin (Mevacor, Altocor, Altoprev), pitavastatin (Livalo, Pitava), pravastatin (Pravachol, Selektine, Lipostat), roosuvastatin (Crestor), simvastatin (Zocor, Lipex), or combinations of a statin or another agent, as ezetimibe/simvastatin. For the treatment of cancer, R-aplexone can be combined or co-administered with any of a variety of anticancer (chemotherapeutic) agents, which will be evident to a skilled worker. These include, e.g., EGFR inhibitors, kinase inhibitors, Taxol related agents, etc.

Combinations may be achieved by administering to a subject, a single composition or pharmacological formulation that includes both agents, or by administering two distinct compositions or formulations, at the same time, wherein one composition includes R-aplexone and the other includes the other agent. Alternatively, the therapy using R-aplexone may precede or follow administration of the other agent(s) by intervals ranging from minutes to weeks.

It also is conceivable that more than one administration of either R-aplexone, or the other agent will be desired. In this regard, various combinations may be employed. By way of illustration, where the R-aplexone is “A” and the other agent is “B”, the following permutations based on 3 and 4 total administrations are exemplary:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are likewise contemplated.

### Surgical Therapeutic Agents

In certain aspects, the secondary therapeutic agent may comprise a surgery of some type, which includes, for example, preventative, diagnostic or staging, curative and palliative surgery. Surgery, and in particular a curative surgery, may be used in conjunction with other therapies, such as the present invention and one or more other agents.

Such surgical therapeutic agents for vascular and cardiovascular diseases and disorders are well known to those of skill in the art, and may comprise, but are not limited to, performing surgery on an organism, providing a cardiovascular mechanical prostheses, angioplasty, coronary artery reperfusion, catheter ablation, providing an implantable cardioverter defibrillator to the subject, mechanical circulatory support or a combination thereof. Non-limiting examples of a mechanical circulatory support that may be used in the present invention comprise an intra-aortic balloon counterpulsation, left ventricular assist device or combination thereof.

### Chemical Definitions

Various chemical definitions related to such compounds are provided as follows.

As used herein, “predominantly one enantiomer” means that the composition contains or the compound is at least about 90, 91, 92, 93, 94, 95.0, 95.1, 95.2, 95.3, 95.4, 95.5, 95.6, 95.7, 95.8, 95.9, 96.0, 96.1, 96.2, 96.3, 96.4, 96.5, 96.6, 96.7, 96.8, 96.9, 97.0 97.1, 97.2, 97.3, 97.4, 97.5, 97.6, 97.7, 97.8, 97.9, 98.0, 98.1, 98.2, 98.3, 98.4, 98.5, 98.6, 98.7, 98.8, 98.9, 99.0, 99.1, 99.2, 99.3, 99.4, 99.5, 99.6, 99.7, 99.8, or 99.9% or any range derivable therein by weight of the one enantiomer. Similarly, the phrase “substantially free of an enantiomer” means that the composition may contain or the compound is no more than about 10, 9, 8, 7, 6, 5, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, or 0.1% or any range derivable therein by weight of the enantiomer in the composition.

As used herein, the term “water soluble” means that the compound dissolves in water at least to the extent of 0.010 mole/liter or is classified as soluble according to literature precedence.

As used herein, the term “nitro” means —NO₂; the term “halo” designates —F, —Cl, —Br or —I; the term “mercapto” means —SH; the term “cyano” means —CN; the term “azido” means —N₃; the term “silyl” means —SiH₃; and the term “hydroxyl” means —OH.

The term “alkyl,” by itself or as part of another substituent, means, unless otherwise stated, a linear (i.e. unbranched) or branched carbon chain, which may be fully saturated, mono- or polyunsaturated. An unsaturated alkyl group is one having one or more double bonds or triple bonds. Saturated alkyl groups include those having one or more carbon-carbon double bonds (alkenyl) and those having one or more carbon-carbon triple bonds (alkynyl). The groups, —CH₃ (Me), —CH₂CH₃ (Et), —CH₂CH₂CH₃ (n-Pr), —CH(CH₃)₂ (iso-Pr), —CH₂CH₂CH₂CH₃ (n-Bu), —CH(CH₃)CH₂CH₃ (sec-butyl), —CH₂CH(CH₃)₂ (iso-butyl), —C(CH₃)₃ (tert-butyl), —CH₂C(CH₃)₃ (neo-pentyl), are all non-limiting examples of alkyl groups.

The term “heteroalkyl,” by itself or in combination with another term, means, unless otherwise stated, a linear or branched chain having at least one carbon atom and at least one heteroatom selected from the group consisting of O, N, S, P, and Si. In certain embodiments, the heteroatoms are selected from the group consisting of O and N. The heteroatom(s) may be placed at any interior position of the heteroalkyl group or at the position at which the alkyl group is attached to the remainder of the molecule. Up to two heteroatoms may be consecutive. The following groups are all non-limiting examples of heteroalkyl groups: trifluoromethyl, —CH₂F, —CH₂Cl, —CH₂Br, —CH₂ OH, —CH₂OCH₃, —CH₂OCH₂CF₃, —CH₂OC(O)CH₃, —CH₂NH₂, —CH₂ NHCH₃, —CH₂ N(CH₃)₂, —CH₂CH₂Cl, —CH₂CH₂OH, CH₂CH₂OC(O)CH₃, —CH₂CH₂ NHCO₂C(CH₃)₃, and —CH₂Si(CH₃)₃.

The terms “cycloalkyl” and “heterocyclyl,” by themselves or in combination with other terms, means cyclic versions of “alkyl” and “heteroalkyl”, respectively. Additionally, for heterocyclyl, a heteroatom can occupy the position at which the heterocycle is attached to the remainder of the molecule.

The term “aryl” means a polyunsaturated, aromatic, hydrocarbon substituent. Aryl groups can be monocyclic or polycyclic (e.g., 2 to 3 rings that are fused together or linked covalently). The term “heteroaryl” refers to an aryl group that contains one to four heteroatoms selected from N, O, and S. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

Various groups are described herein as substituted or unsubstituted (i.e., optionally substituted). Optionally substituted groups may include one or more substituents independently selected from: halogen, nitro, cyano, hydroxy, amino, mercapto, formyl, carboxy, oxo, carbamoyl, substituted or unsubstituted alkyl, substituted or unsubstituted heteroalkyl, alkoxy, alkylthio, alkylamino, (alkyl)₂amino, alkylsulfinyl, alkylsulfonyl, arylsulfonyl, substituted or unsubstituted cycloalkyl, substituted or unsubstituted heterocyclyl, substituted or unsubstituted aryl, and substituted or unsubstituted heteroaryl. In certain aspects, the optional substituents may be further substituted with one or more substituents independently selected from: halogen, nitro, cyano, hydroxy, amino, mercapto, formyl, carboxy, carbamoyl, unsubstituted alkyl, unsubstituted heteroalkyl, alkoxy, alkylthio, alkylamino, (alkyl)₂amino, alkylsulfinyl, alkylsulfonyl, arylsulfonyl, unsubstituted cycloalkyl, unsubstituted heterocyclyl, unsubstituted aryl, or unsubstituted heteroaryl. Exemplary optional substituents include, but are not limited to: —OH, oxo (═O), —Cl, —F, Br, C₁₋₄alkyl, phenyl, benzyl, —NH₂, —NH(C₁₋₄alkyl), —N(C₁₋₄alkyl)₂, —NO₂, —S(C₁₋₄alkyl), —SO₂(C₁₋₄alkyl), —CO₂(C₁₋₄alkyl), and —O(C₁₋₄alkyl).

The term “alkoxy” means a group having the structure —OR′, where R′ is an optionally substituted alkyl or cycloalkyl group. The term “heteroalkoxy” similarly means a group having the structure —OR, where R is a heteroalkyl or heterocyclyl.

The term “amino” means a group having the structure —NR′R″, where R′ and R″ are independently hydrogen or an optionally substituted alkyl, heteroalkyl, cycloalkyl, or heterocyclyl group. The term “amino” includes primary, secondary, and tertiary amines.

The term “oxo” as used herein means an oxygen that is double bonded to a carbon atom.

The term “alkylsulfonyl” as used herein means a moiety having the formula —S(O₂)—R′, where R′ is an alkyl group. R′ may have a specified number of carbons (e.g. “C₁₋₄ alkylsulfonyl”)

The term “pharmaceutically acceptable salts,” as used herein, refers to salts of compounds of this invention that are substantially non-toxic to living organisms. Typical pharmaceutically acceptable salts include those salts prepared by reaction of a compound of this invention with an inorganic or organic acid, or an organic base, depending on the substituents present on the compounds of the invention.

Non-limiting examples of inorganic acids which may be used to prepare pharmaceutically acceptable salts include: hydrochloric acid, phosphoric acid, sulfuric acid, hydrobromic acid, hydroiodic acid, phosphorous acid and the like. Examples of organic acids which may be used to prepare pharmaceutically acceptable salts include: aliphatic mono- and dicarboxylic acids, such as oxalic acid, carbonic acid, citric acid, succinic acid, phenyl-heteroatom-substituted alkanoic acids, aliphatic and aromatic sulfuric acids and the like. Pharmaceutically acceptable salts prepared from inorganic or organic acids thus include hydrochloride, hydrobromide, nitrate, sulfate, pyrosulfate, bisulfate, sulfite, bisulfate, phosphate, monohydrogenphosphate, dihydrogenphosphate, metaphosphate, pyrophosphate, hydroiodide, hydro fluoride, acetate, propionate, formate, oxalate, citrate, lactate, p-toluenesulfonate, methanesulfonate, maleate, and the like.

Suitable pharmaceutically acceptable salts may also be formed by reacting the agents of the invention with an organic base such as methylamine, ethylamine, ethanolamine, lysine, ornithine and the like. Pharmaceutically acceptable salts include the salts formed between carboxylate or sulfonate groups found on some of the compounds of this invention and inorganic cations, such as sodium, potassium, ammonium, or calcium, or such organic cations as isopropylammonium, trimethylammonium, tetramethylammonium, and imidazolium.

It should be recognized that the particular anion or cation forming a part of any salt of this invention is not critical, so long as the salt, as a whole, is pharmacologically acceptable.

Additional examples of pharmaceutically acceptable salts and their methods of preparation and use are presented in Handbook of Pharmaceutical Salts: Properties, Selection and Use (2002), which is incorporated herein by reference.

An “isomer” of a first compound is a separate compound in which each molecule contains the same constituent atoms as the first compound, but where the configuration of those atoms in three dimensions differs. Unless otherwise specified, the compounds described herein are meant to encompass their isomers as well. A “stereoisomer” is an isomer in which the same atoms are bonded to the same other atoms, but where the configuration of those atoms in three dimensions differs. “Enantiomers” are stereoisomers that are mirror images of each other, like left and right hands. “Diastereomers” are stereoisomers that are not enantiomers.

It is contemplated that any embodiment discussed in this specification can be implemented with respect to any method or composition of the invention, and vice versa. Furthermore, compositions of the invention can be used to achieve methods of the invention.

### EXAMPLES

The following examples as well as the figures are included to demonstrate non-limiting embodiments of the invention. It should be appreciated by those of skill in the art that the techniques disclosed in the examples or figures represent non-limiting techniques discovered by the inventors to function well in the practice of the invention. However, those of skill in the art should, in light of the present disclosure, appreciate that many changes can be made in the specific embodiments which are disclosed and still obtain a like or similar result without departing from the spirit and scope of the invention.

### Example I

### Annulation Production and Products

Phosphine-Catalyzed [4+2] Allenoate-Imine Annulation

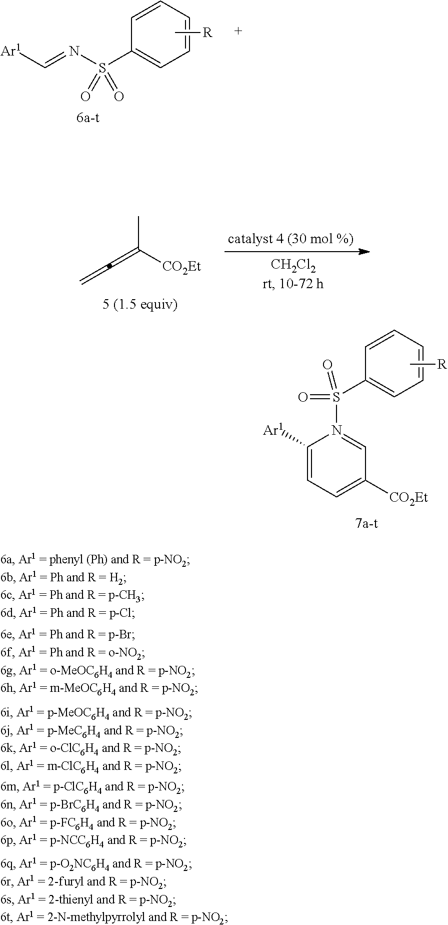

### Catalyst 4

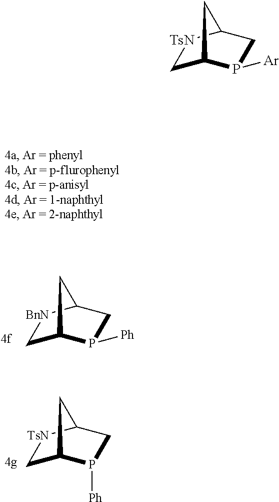

0.1 mmol imine 6a-f, 10 mg of additive and 0.3 equivalent of the catalyst 4 were weighed and added to a flask in a glove box. Distilled CH₂Cl₂ (1.5 mL) was added via syringe under argon. Allenoate 5 (1.5 equiv) was added dropwise to the reaction mixture via syringe. The flask was capped with a Teflon cap and sealed with Teflon tape and parafilm. The contents were stirred at room temperature for three days. The crude reaction mixture was loaded directly onto a silica gel column and purified chromatographically (hexane/EtOAc, 3:1).

### 1.0 Mmol-Scale Reaction

A flame-dried round bottom flask equipped with a magnetic stir-bar was charged with imine 6c (259 mg, 1.0 equiv), 4 Å molecular sieves (100 mg) and catalyst 4c (112 mg, 0.3 equiv) in a glove box. Distilled CH₂Cl₂ was added via syringe under argon, followed by acetic acid (5.7 μL, 0.1 equiv). Allenoate 5 (189 mg, 1.5 equiv) was then added dropwise to the reaction mixture via syringe. The flask was capped with a Teflon cap, sealed with Teflon tape and parafilm, then allowed to stir for 72 h at room temperature. Upon completion, the mixture was concentrated and purified chromatographically as described above to give 230 mg (60% yield, 93% ee) of annulation product 7c.

TABLE 1
Annulations products 7

| entry | 6 | 4 | additive | ratio^{a} | Yield (%)^{b} | ee (%)^{c, d} |
|---|---|---|---|---|---|---|
| 1 | 6a | 4a | none | 1:0.03 | 36 | 80 |
| 2 | 6a | 4a | MgSO₄ | 1:0.03 | 42 | 77 |
| 3 | 6a | 4a | Na₂CO₃ | 1:0.03 | 54 | 75 |
| 4 | 6a | 4a | 4 Å MS | 1:0.03 | 89 | 79 |
| 5 | 6c | 4a | 4 Å MS | 1:0.22 | 78 | 89 |
| 6 | 6a | 4b | 4 Å MS | 1:0.02 | 71 | 77 |
| 7 | 6a | 4c | 4 Å MS | >1:0.01 | 89 | 77 |
| 8 | 6a | 4d | 4 Å MS | 1:0.08 | 54 | 79 |
| 9 | 6a | 4e | 4 Å MS | 1:0.03 | 81 | 81 |
| 10 | 6a | 4f | 4 Å MS | >1:0.01 | 84 | 73 |
| 11 | 6a | 4g | 4 Å MS | 1:0.01 | 82 | −48 |
| 12^{e} | 6a | 4c | 4 Å MS | 1:0.01 | 70 | 95 |
| 13^{e} | 6b | 4c | 4 Å MS | 1:0.04 | 50 | 97 |
| 14^{e} | 6c | 4c | 4 Å MS | 1:0.05 | 56 | 98 |
| 15^{e} | 6d | 4c | 4 Å MS | 1:0.14 | 67 | 91 |
| 16^{e} | 6e | 4c | 4 Å MS | 1:0.02 | 46 | 94 |
| 17^{e} | 6f | 4c | 4 Å MS | 1:0.05 | 29 | 97 |

^{a}Regioisomeric ratio of γ:β′ adducts, determined from the ¹H NMR spectrum of the crude reaction product.
^{b}Isolated yield after column chromatography.
^{c}Determined through supercritical fluid chromatography (SFC) using an AS or OJ-H column.
^{d}The ee of the major product.
^{e}AcOH (10 mol %) was added.

### Analytical Data for Annulation Products 7

Flash column chromatography was performed using E. Merck silica gel 60 (230-400 mesh) and compressed air. IR spectra were recorded on a Perkin-Elmer pargon 1600 FT-IR spectrometer. NMR spectra were obtained on AV-500, DRX-500, AV-300, or ARX-400 instruments as indicated, calibrated using residual CHCl₃ as the internal reference (7.26 ppm for ¹H NMR; 77.00 ppm for ¹³C NMR). Data for ¹H NMR spectra are reported as follows: chemical shift (S ppm), multiplicity, coupling constant (Hz), and integration. The following abbreviations are used for the multiplicities: s=singlet; d=doublet; t=triplet; q=quartet;m=multiplet; br=broad. High-resolution mass spectrometry (HRMS) was performed using a Waters LCT Premier XE time-of-flight instrument controlled by Mass Lynx 4.1 software; mass-analyzed laser desorption/ionization (MALDI) mass spectra were recorded using an AB/PerSpective DE-STR TOF instrument with 2,5-dihydroxybenzoic acid as the matrix. Optical rotations were determined using an Autopol IV polarimeter and a 50-mm cell at concentrations and temperatures indicated.

Enantiomeric excess for the annulation products (7) were determined on a Mettler Toledo supercritical fluid chromatography (SFC) system using a chiral AS column (10% MeOH as a modifier; 2 mL/min). The ee value for compound 7t was determined using a Shimadzu CBM Lite system equipped with a chiral AD-H column, eluting with 10% isopropyl alcohol in hexanes at a flow rate of 1.5 mL/min. Enantiomeric excess for aplexone (3) were determined using the above mentioned SFC instrument using a chiral OJ-H column (10% MeOH as a modifier; 2 mL/min).

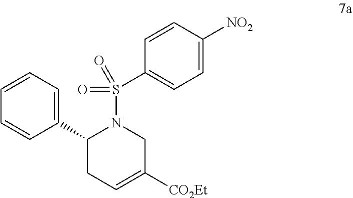 [SMILES: CCOC(=O)C1=CC[C@H](c2ccccc2)N(S(=O)(=O)c2ccc([N+](=O)[O-])cc2)C1]

### Ethyl (R)-1-((4-nitrophenyl)sulfonyl)-6-phenyl-1,2,5,6-tetrahydropyridine-3-carboxylate (7a)

29.1 mg (70% yield); 95% ee; [α]²⁰_{D} −28.2° (c 1.00, CHCl₃); IR (film) vₘₐₓ 2984, 1708, 1349 cm⁻¹; ¹H (500 MHz, CDCl₃) δ 8.29 (d, J=8.9 Hz, 2H), 7.96 (d, J=8.9 Hz, 2H), 7.29-7.26 (m, 3H), 7.21-7.19 (m, 2H), 7.06-7.05 (m, 1H), 5.40 (d, J=6.9 Hz, 1H), 4.51 (d, J=18.0 Hz, 1H), 4.21 (q, J=7.2 Hz, 2H), 3.53-3.48 (m, 1H), 2.74-2.56 (m, 2H), 1.29 (t, J=7.2 Hz, 3H); ¹³C (125 MHz, CDCl₃) δ 164.3, 149.9, 145.9, 137.6, 135.9, 128.8, 128.3, 128.2, 127.3, 127.1, 124.3, 61.1, 52.8, 39.9, 27.8, 14.2; HRMS (ESI) Calculated for [M+H]⁺, C₂₀H₂₁N₂O₆S, m/z 417.1120, found 417.1112.

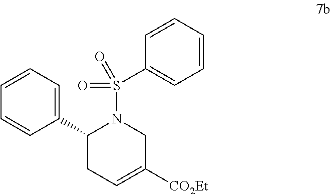 [SMILES: CCOC(=O)C1=CC[C@H](c2ccccc2)N(S(=O)(=O)c2ccccc2)C1]

### Ethyl (R)-6-phenyl-1-(phenylsulfonyl)-1,2,5,6-tetrahydropyridine-3-carboxylate (7b)

18.6 mg (50% yield); 97% ee; [α]²⁰_{D} −43.6° (c 1.00, CHCl₃); IR (film) vₘₐₓ 2982, 1710, 1350 cm⁻¹; ¹H (500 MHz, CDCl₃) δ 7.81 (dd, J=8.1, 1.2 Hz, 2H), 7.56 (t, J=7.5 Hz, 1H), 7.47 (t, J=7.5 Hz, 2H), 7.29-7.22 (m, 5H), 7.02-7.01 (m, 1H), 5.36 (d, J=6.8 Hz, 1H), 4.51 (d, J=18.6 Hz, 1H), 4.21 (q, J=7.2 Hz, 2H), 3.49-3.43 (m, 1H), 2.69-2.49 (m, 2H), 1.28 (t, J=7.2 Hz, 3H); ¹³C (125 MHz, CDCl₃) δ 164.6, 140.3, 138.0, 136.1, 132.7, 129.1, 128.6, 127.9, 127.6, 127.2, 126.9, 60.9, 52.2, 39.6, 27.1, 14.2; HRMS (ESI) Calculated for [M+H]⁺, C₂₀H₂₂NO₄S, m/z 372.1270, found 372.1265.

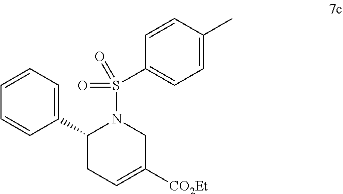 [SMILES: CCOC(=O)C1=CC[C@H](c2ccccc2)N(S(=O)(=O)c2ccc(C)cc2)C1]

### Ethyl (R)-6-phenyl-1-tosyl-1,2,5,6-tetrahydropyridine-3-carboxylate (7c)

21.6 mg (56% yield); as a white solid: mp 145-146° C.; 98% ee; [α]²⁰_{D} −49.0° (c 1.00, CHCl₃); IR (film) vₘₐₓ 2981, 1708, 1659, 1598, 1495, 1449, 1340, 1263, 1161, 1099, 958, 727, 667 cm⁻¹; ¹H NMR (500 MHz, CDCl₃) δ 7.66 (d, J=8.2 Hz, 2H), 7.28-7.21 (m, 7H), 6.99 (m, 1H), 5.33 (d, J=6.8 Hz, 1H), 4.45 (br d, J=18.6 Hz, 1H), 4.14 (q, J=7.2 Hz, 2H), 3.43 (dm, J=18.6 Hz, 1H), 2.64 (ddd, J=19.4, 5.7, 2.7 Hz, 1H), 2.53-2.46 (m, 1H), 2.39 (s, 3H), 1.24 (t, J=7.2 Hz, 3H); ¹³C NMR (100 MHz, CDCl₃) δ 164.6, 143.4, 138.1, 137.4, 136.1, 129.6 (2C), 128.5 (2C), 127.8, 127.6, 127.2 (2C), 127.0 (2C), 60.7, 52.1, 39.5, 26.9, 21.5, 14.1; HRMS (EI) calcd for C₂₁H₂₃NO₄S [M⁺] 385.1348, found 385.1343.

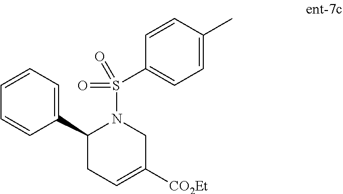 [SMILES: CCOC(=O)C1=CC[C@@H](c2ccccc2)N(S(=O)(=O)c2ccc(C)cc2)C1]

### Ethyl (S)-6-phenyl-1-tosyl-1,2,5,6-tetrahydropyridine-3-carboxylate (ent-7c)

21.5 mg (56% yield); 98% ee; [α]²⁰_{D} +49.0° (c 1.00, CHCl₃). This annulation product was synthesized as described in the general [4+2] procedure, however ent-4c was used as the catalyst. The NMR spectral data is identical to that of the R-enantiomer, 7c.

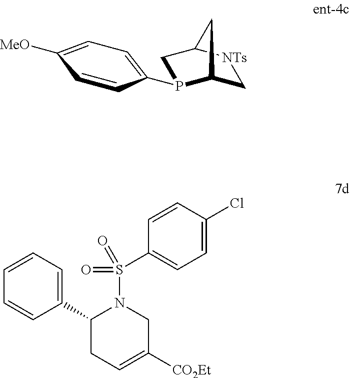

### Ethyl (R)-1-((4-chlorophenyl)sulfonyl)-6-phenyl-1,2,5,6-tetrahydropyridine-3-carboxylate (7d)

27.1 mg (67% yield); 91% ee; [α]²⁰_{D} +1.0° (c 1.00, CHCl₃). IR (film) vₘₐₓ 2979, 1706, 1531, 1324 cm⁻¹; ¹H (400 MHz, CDCl₃) δ 7.93 (d, J=8.7 Hz, 2H), 7.63 (d, J=8.7 Hz, 2H), 7.49-7.41 (m, 5H), 7.25-7.23 (m, 1H), 5.56 (d, J=6.7 Hz, 1H), 4.68 (d, J=18.5 Hz, 1H), 4.40 (q, J=7.2 Hz, 2H), 3.70-3.63 (m, 1H), 2.92-2.72 (m, 2H), 1.49 (t, J=7.2 Hz, 3H); ¹³C (125 MHz, CDCl₃) δ 164.5, 139.1, 138.8, 137.9, 136.0, 129.4, 128.7, 128.4, 128.0, 127.4, 127.2, 60.9, 52.4, 39.7, 27.4, 14.2; HRMS (ESI) Calculated for [M+H]⁺, C₂₀H₂₁ClNO₄S, m/z 406.0880, found 406.0879.

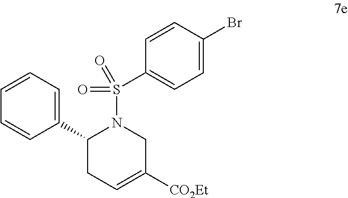 [SMILES: CCOC(=O)C1=CC[C@H](c2ccccc2)N(S(=O)(=O)c2ccc(Br)cc2)C1]

### Ethyl (R)-1-((4-bromophenyl)sulfonyl)-6-phenyl-1,2,5,6-tetrahydropyridine-3-carboxylate (7e)

20.6 mg (46% yield); 94% ee; [α]²⁰_{D} +1.4° (c 1.00, CHCl₃); IR (film) vₘₐₓ 2978, 1707, 1326 cm⁻¹; ¹H NMR (500 MHz, CDCl₃) δ 7.65 (d, J=8.6 Hz, 2H), 7.58 (d, J=8.6 Hz, 2H), 7.33-7.27 (m, 3H), 7.22 (d, J=6.6 Hz, 2H), 7.05 (d, J=3.4 Hz, 1H), 5.35 (d, J=6.8 Hz, 1H), 4.45 (d, J=18.4 Hz, 1H), 4.18 (q, J=7.1 Hz, 2H), 3.46 (dd, J=2.2, 18.5 Hz, 1H), 2.69 (dd, J=2.9, 19.5 Hz, 1H), 2.56 (dd, J=2.7, 19.4 Hz, 1H), 1.27 (t, J=7.1 Hz, 3H); ¹³C NMR (125 MHz, CDCl₃) δ 164.4, 139.2, 137.8, 135.9, 132.2, 128.6, 128.4, 127.9, 127.5, 127.3, 127.0, 60.8, 52.3, 39.6, 27.3, 14.1; HRMS (ESI) Calculated for [M−H]⁺, C₂₀H₁₉BrNO₄S, m/z 448.0218, found 448.0220. C₂₀H₁₉BrNO₄S

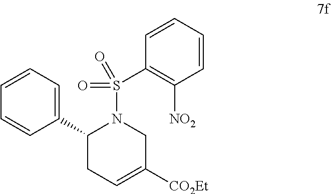 [SMILES: CCOC(=O)C1=CC[C@H](c2ccccc2)N(S(=O)(=O)c2ccccc2[N+](=O)[O-])C1]

### Ethyl (R)-1-((2-nitrophenyl)sulfonyl)-6-phenyl-1,2,5,6-tetrahydropyridine-3-carboxylate (7f)

12.1 mg (29% yield); 97% ee; [α]²⁰_{D} −24.4° (c 1.00, CHCl₃); IR (film) vₘₐₓ 2977, 1709, 1530, 1164 cm⁻¹; ¹H NMR (500 MHz, CDCl₃) δ 8.06-8.03 (m, 1H), 7.73-7.65 (m, 3H), 7.33-7.26 (m, 5H), 7.14 (dd, J=1.9, 5.3 Hz, 1H), 5.41 (d, J=6.4 Hz, 1H), 4.37 (dd, J=0.9, 18.4 Hz, 1H), 4.17 (q, J=7.1 Hz, 2H), 3.58 (ddt, J=2.5, 4.2, 18.3 Hz, 1H), 2.89 (ddd, J=2.2, 9.4, 19.5 Hz, 1H), 2.80 (ddd, J=1.4, 4.1, 19.4 Hz, 1H), 1.27 (t, J=7.2 Hz, 3H); ¹³C NMR (125 MHz, CDCl₃) δ 164.4, 147.7, 137.5, 136.2, 133.6, 133.4, 131.9, 130.8, 128.6, 127.9, 127.3, 127.1, 124.4, 60.8, 52.7, 40.0, 27.6, 14.0; HRMS (ESI) Calculated for [M+H]⁺, C₂₀H₂₁N₂O₆S, m/z 417.1120, found 417.1129

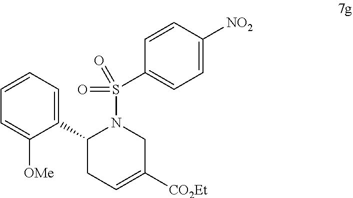 [SMILES: CCOC(=O)C1=CC[C@H](c2ccccc2OC)N(S(=O)(=O)c2ccc([N+](=O)[O-])cc2)C1]

### Ethyl (R)-6-(2-methoxyphenyl)-1-((4-nitrophenyl)sulfonyl)-1,2,5,6-tetrahydropyridine-3-carboxylate (7g)

36.1 mg (81% yield); 95% ee; [α]²⁰_{D}=+14.0° (c 1.00, CHCl₃); IR (film) vₘₐₓ 2986, 1712, 1531, 1164 cm⁻¹; ¹H (500 MHz, CDCl₃) δ 8.20 (d, J=8.8 Hz, 2H), 7.89 (d, J=8.8 Hz, 2H), 7.22-7.19 (m, 1H), 7.11 (d, J=2.7 Hz, 1H), 6.94 (dd, J=1.2, 7.6 Hz, 1H), 6.79 (d, J=8.2 Hz, 1H), 6.75 (t, J=7.5 Hz, 1H), 5.75 (d, J=7.1 Hz, 1H), 4.52 (d, J=18.0 Hz, 1H), 4.23 (q, J=7.1 Hz, 2H), 3.69 (s, 3H), 3.68-3.67 (m, 1H), 2.87-2.56 (m, 2H), 1.30 (t, J=7.1 Hz, 3H); ¹³C (125 MHz, CDCl₃) δ 164.6, 156.5, 149.7, 146.2, 137.0, 129.5, 128.2, 127.3, 127.0, 126.8, 123.8, 120.3, 110.6, 60.9, 55.2, 47.3, 40.7, 29.5, 14.2; HRMS (ESI) Calculated for [M+H]⁺, C₂₀H₂₃N₂O₆S, m/z 447.1226, found 447.1215.

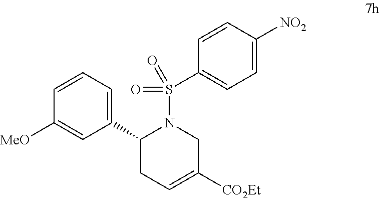 [SMILES: CCOC(=O)C1=CC[C@H](c2cccc(OC)c2)N(S(=O)(=O)c2ccc([N+](=O)[O-])cc2)C1]

### Ethyl (R)-6-(3-methoxyphenyl)-1-((4-nitrophenyl)sulfonyl)-1,2,5,6-tetrahydropyridine-3-carboxylate (7h)

29.4 mg (66% yield); 97% ee; [α]²⁰_{D} −18.4° (c 1.00, CHCl₃); IR (film) vₘₐₓ 2984, 1708, 1528, 1165 cm⁻¹; ¹H (500 MHz, CDCl₃) δ 8.28 (d, J=8.8 Hz, 2H), 7.96 (d, J=8.8 Hz, 2H), 7.18 (t, J=7.9 Hz, 1H), 7.04-7.03 (m, 1H), 6.79-6.75 (m, 2H), 6.71 (br s, 1H), 5.35 (d, J=6.8 Hz, 1H), 4.51 (d, J=18.3 Hz, 1H), 4.19 (q, J=7.2 Hz, 2H), 3.74 (s, 3H), 3.57-3.52 (m, 1H), 2.72-2.56 (m, 2H), 1.29 (t, J=7.2 Hz, 3H); ¹³C (125 MHz, CDCl₃) δ 164.3, 159.8, 149.9, 145.9, 139.2 135.9, 129.8, 128.2, 127.2, 124.3, 119.2, 113.6, 112.9, 61.1, 55.2, 52.7, 40.0, 27.9, 14.2; HRMS (ESI) Calculated for [M+H]⁺, C₂₀H₂₃N₂O₆S, m/z 447.1226, found 447.1233.

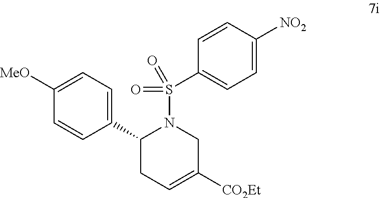 [SMILES: CCOC(=O)C1=CC[C@H](c2ccc(OC)cc2)N(S(=O)(=O)c2ccc([N+](=O)[O-])cc2)C1]

### Ethyl (R)-6-(4-methoxyphenyl)-1-((4-nitrophenyl)sulfonyl)-1,2,5,6-tetrahydropyridine-3-carboxylate (7i)

28.0 mg (63% yield); 96% ee; [α]²⁰_{D} −18.4° (c 1.00, CHCl₃); IR (film) vₘₐₓ 2984, 1709, 1531, 1349, 1253, 1164 cm⁻¹; ¹H (500 MHz, CDCl₃) δ 8.29 (d, J=8.9 Hz, 2H), 7.96 (d, J=8.9 Hz, 2H), 7.13 (d, J=8.6 Hz, 2H), 7.04-7.03 (m, 1H), 6.79 (d, J=8.6 Hz, 2H), 5.35 (d, J=6.7 Hz, 1H), 4.48 (d, J=18.3 Hz, 1H), 4.20 (q, J=7.2 Hz, 2H), 3.76 (s, 3H), 3.51-3.45 (m, 1H), 2.69-2.53 (m, 2H), 1.29 (t, J=7.2 Hz, 3H); ¹³C (125 MHz, CDCl₃) δ 164.4, 159.4, 149.9, 146.1, 136.0, 129.0, 128.4, 128.2, 127.3, 124.3, 114.1, 61.1, 55.3, 52.3, 39.8, 27.9, 14.2; HRMS (ESI) Calculated for [M−H]⁺, C₂₀H₂₃N₂O₆S, m/z 445.1069, found 445.1098.

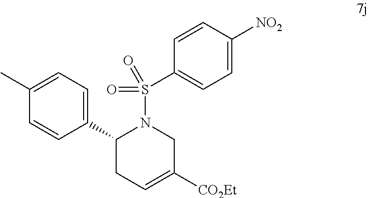 [SMILES: CCOC(=O)C1=CC[C@H](c2ccc(C)cc2)N(S(=O)(=O)c2ccc([N+](=O)[O-])cc2)C1]

### Ethyl (R)-1-((4-nitrophenyl)sulfonyl)-6-(p-tolyl)-1,2,5,6-tetrahydropyridine-3-carboxylate (7j)

27.5 mg (64% yield); 98% ee; [α]²⁰_{D} −21.2° (c 1.00, CHCl₃); IR (film) vₘₐₓ 1708, 1530, 1349, 1164 cm⁻¹; ¹H (500 MHz, CDCl₃) δ 8.29 (d, J=8.9 Hz, 2H), 7.96 (d, J=8.9 Hz, 2H), 7.07 (s, 4H), 7.05-7.04 (m, 1H), 5.36 (d, J=6.8 Hz, 1H), 4.49 (d, J=18.3 Hz, 1H), 4.20 (q, J=7.2 Hz, 2H), 3.52-3.47 (m, 1H), 2.71-2.55 (m, 2H), 2.30 (s, 3H), 1.28 (t, J=7.2 Hz, 3H); ¹³C (125 MHz, CDCl₃) δ 164.4, 149.9, 146.1, 138.1, 136.1, 134.5, 129.4, 128.2, 127.3, 127.0, 124.3, 61.0, 52.6, 39.8, 27.8, 21.0, 14.2; HRMS (ESI) Calculated for [M+H]⁺, C₂₁H₂₃N₂O₆S, m/z 431.1277, found 431.1277.

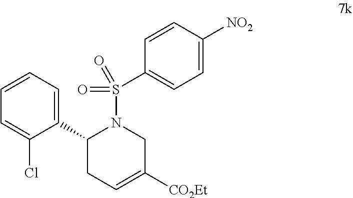 [SMILES: CCOC(=O)C1=CC[C@H](c2ccccc2Cl)N(S(=O)(=O)c2ccc([N+](=O)[O-])cc2)C1]

### Ethyl (R)-6-(2-chlorophenyl)-1-((4-nitrophenyl)sulfonyl)-1,2,5,6-tetrahydropyridine-3-carboxylate (7k)

13.0 mg (29% yield; 74% ee); [α]²⁰_{D} +26.8° (c 1.00, CHCl₃); IR (film) vₘₐₓ 2979, 1710, 1530, 1350, 1262, 1168, 1095 cm⁻¹; ¹H (500 MHz, CDCl₃) δ 8.20 (d, J=8.8 Hz, 2H), 7.91 (d, J=8.8 Hz, 2H), 7.34 (dd, J=8.8, 1.1 Hz, 1H), 7.19 (dt, J=7.8, 1.5 Hz, 1H), 7.10-7.07 (m, 1H), 7.06 (dt, J=7.8, 1.1 Hz, 1H), 6.99 (dd, J=7.8, 1.4 Hz, 1H), 5.76 (dd, J=7.2, 1.6 Hz, 1H), 4.49 (d, J=18.1 Hz, 1H), 4.25 (q, J=7.1 Hz, 2H), 3.81 (dd, J=18.1, 2.8 Hz, 1H), 2.93-2.89 (m, 1H), 2.66-2.61 (m, 1H), 1.32 (t, J=7.1 Hz, 3H); ¹³C (125 MHz, CDCl₃) δ 164.3, 149.9, 145.0, 136.5, 136.1, 133.5, 130.2, 129.5, 128.5, 127.6, 127.5, 126.9, 123.9, 61.1, 50.4, 41.2, 29.5, 14.2; HRMS (ESI) Calculated for [M−H]⁺, C₂₀H₁₈ClN₂O₆S, m/z 449.0574, found 449.0584.

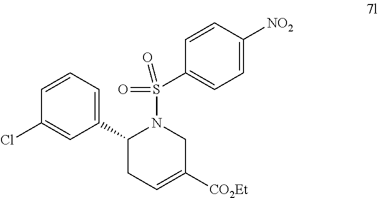 [SMILES: CCOC(=O)C1=CC[C@H](c2cccc(Cl)c2)N(S(=O)(=O)c2ccc([N+](=O)[O-])cc2)C1]

### Ethyl (R)-6-(3-chlorophenyl)-1-((4-nitrophenyl)sulfonyl)-1,2,5,6-tetrahydropyridine-3-carboxylate (71)

22.5 mg (50% yield; 94% ee); [α]²⁰_{D}=−30.8° (c 1.00, CHCl₃); IR (film) vₘₐₓ 2981, 1712, 1530, 1349, 1165 cm⁻¹; ¹H (500 MHz, CDCl₃) δ 8.32 (d, J=8.8 Hz, 2H), 7.98 (d, J=8.8 Hz, 2H), 7.26-7.21 (m, 2H), 7.12-7.11 (m, 2H), 7.04-7.02 (m, 1H), 5.36 (d, J=6.8 Hz, 1H), 4.53 (d, J=18.4 Hz, 1H), 4.22 (q, J=7.1 Hz, 2H), 3.54-3.49 (m, 1H), 2.72-2.54 (m, 2H), 1.30 (t, J=7.1 Hz, 3H); ¹³C (125 MHz, CDCl₃) δ 164.2, 150.1, 145.8, 139.5, 135.45 135.4, 134.8, 130.2, 128.5, 128.1, 127.4, 125.2, 124.4, 61.2, 52.3, 40.0, 27.5, 14.2; HRMS (ESI) Calculated for [M+H]⁺, C₂₀H₂₀ClN₂O₆S, m/z 451.0731, found 451.0721.

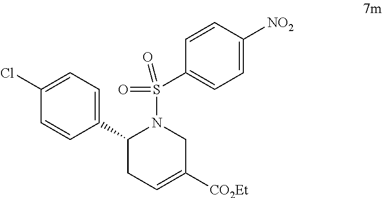 [SMILES: CCOC(=O)C1=CC[C@H](c2ccc(Cl)cc2)N(S(=O)(=O)c2ccc([N+](=O)[O-])cc2)C1]

### Ethyl (R)-6-(4-chlorophenyl)-1-((4-nitrophenyl)sulfonyl)-1,2,5,6-tetrahydropyridine-3-carboxylate (7m)

23.7 mg (53% yield); 97% ee; [α]²⁰_{D} −31.6° (c 1.00, CHCl₃); IR (film) vₘₐₓ 2982, 1712, 1531, 1349, 1165, 1099 cm⁻¹; ¹H (500 MHz, CDCl₃) δ 8.32 (d, J=8.9 Hz, 2H), 7.98 (d, J=8.9 Hz, 2H), 7.28 (d, J=8.5 Hz, 2H), 7.19 (d, J=8.5 Hz, 2H), 7.02-7.01 (m, 1H), 5.38 (d, J=6.8 Hz, 1H), 4.48 (d, J=18.6 Hz, 1H), 4.20 (q, J=7.2 Hz, 2H), 3.50-3.44 (m, 1H), 2.71-2.65 (m, 1H), 2.56-2.49 (m, 1H), 1.29 (t, J=7.2 Hz, 3H); ¹³C (125 MHz, CDCl₃) δ 164.2, 150.0, 145.9, 136.0, 135.5, 134.2, 128.9, 128.6, 128.1, 127.4, 124.5, 61.1, 52.1, 39.8, 27.3, 14.1; HRMS (ESI) Calculated for [M−H]⁺, C₂₀H₁₈ClN₂O₆S, m/z 449.0574, found 449.0576.

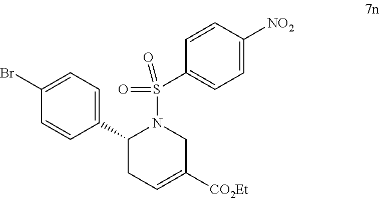 [SMILES: CCOC(=O)C1=CC[C@H](c2ccc(Br)cc2)N(S(=O)(=O)c2ccc([N+](=O)[O-])cc2)C1]

### Ethyl (R)-6-(4-bromophenyl)-1-((4-nitrophenyl)sulfonyl)-1,2,5,6-tetrahydropyridine-3-carboxylate (7n)

25.1 mg (51% yield); 97% ee; [α]²⁰_{D} −16.0° (c 1.00, CHCl₃); IR (film) vₘₐₓ 2979, 1710, 1531, 1350, 1266, 1165, 1100 cm 1; ¹H (300 MHz, CDCl₃) δ 8.33 (d, J=9.0 Hz, 2H), 7.99 (d, J=9.0 Hz, 2H), 7.44 (d, J=8.6 Hz, 2H), 7.13 (d, J=8.6 Hz, 2H), 7.03-7.00 (m, 1H), 5.36 (d, J=6.5 Hz, 1H), 4.49 (d, J=18.9 Hz, 1H), 4.22 (q, J=7.1 Hz, 2H), 3.52-3.43 (m, 1H), 2.72-2.47 (m, 2H), 1.28 (t, J=7.1 Hz, 3H); ¹³C (125 MHz, CDCl₃) δ 164.2, 150.1, 145.9, 136.5, 135.4, 132.0, 128.9, 128.1, 127.4, 124.5, 122.4, 61.2, 52.2, 39.8, 27.3, 14.2; HRMS (ESI) Calculated for [M−H]⁺, C₂₀H₁₈BrN₂O₆S, m/z 493.0069, found 493.0069.

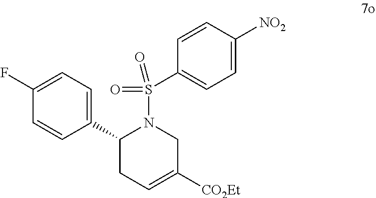 [SMILES: CCOC(=O)C1=CC[C@H](c2ccc(F)cc2)N(S(=O)(=O)c2ccc([N+](=O)[O-])cc2)C1]

### Ethyl (R)-6-(4-fluorophenyl)-1-((4-nitrophenyl)sulfonyl)-1,2,5,6-tetrahydropyridine-3-carboxylate (7o)

27.6 mg (64% yield); 96% ee; [α]²⁰_{D} −42.0° (c 1.00, CHCl₃); IR (film) vₘₐₓ 2982, 1711, 1531, 1350, 1266, 1166, 1099 cm⁻¹; ¹H (500 MHz, CDCl₃) δ 8.32 (d, J=8.8 Hz, 2H), 7.98 (d, J=8.8 Hz, 2H), 7.23 (dd, J=5.3, 8.6 Hz, 2H), 7.04-7.02 (m, 1H), 7.00 (t, J=8.6 Hz, 2H), 5.38 (d, J=6.8 Hz, 1H), 4.48 (d, J=18.4 Hz, 1H), 4.20 (q, J=7.2 Hz, 2H), 3.50-3.46 (m, 1H), 2.70-2.52 (m, 2H), 1.29 (t, J=7.2 Hz, 3H); ¹³C (125 MHz, CDCl₃) δ 164.2, 162.4 (d, J_{C-F}=246 Hz), 150.0, 146.0, 135.6, 133.3, 129.0, 128.1, 124.7, 115.7, 115.6, 61.2, 52.1, 39.7, 27.5, 14.1; HRMS (ESI) Calculated for [M−H]⁺, C₂₀H₁₈FN₂O₆S, m/z 433.0870, found 433.0862.

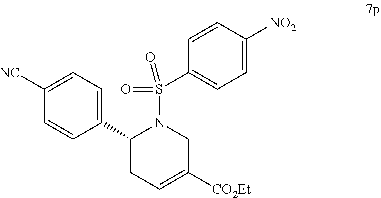 [SMILES: CCOC(=O)C1=CC[C@H](c2ccc(C#N)cc2)N(S(=O)(=O)c2ccc([N+](=O)[O-])cc2)C1]

### Ethyl (R)-6-(4-cyanophenyl)-1-((4-nitrophenyl)sulfonyl)-1,2,5,6-tetrahydropyridine-3-carboxylate (7p)

22.4 mg (51% yield); 90% ee; [α]²⁰_{D} −20.8° (c 1.00, CHCl₃); IR (film) vₘₐₓ 2980, 1710, 1531, 1350, 1267, 1165, 1100; ¹H (400 MHz, CDCl₃) δ 8.24 (d, J=8.8 Hz, 2H), 7.91 (d, J=8.8 Hz, 2H), 7.53 (d, J=8.3 Hz, 2H), 7.32 (d, J=8.3 Hz, 2H), 6.91-6.89 (m, 1H), 5.34 (d, J=6.6 Hz, 1H), 4.38 (d, J=18.7 Hz, 1H), 4.09 (q, J=7.1 Hz, 2H), 3.41-3.35 (m, 1H), 2.66-2.39 (m, 2H), 1.18 (t, J=7.1 Hz, 3H); ¹³C (100 MHz, CDCl₃) δ 164.0, 150.2, 145.7, 142.8, 135.0, 132.7, 128.1, 127.9, 127.5, 124.6, 118.2, 112.3, 61.2, 52.3, 39.9, 26.8, 14.1; HRMS (ESI) Calculated for [M−H]⁺, C₂₁H₁₈N₃O₆S, m/z 440.0916, found 440.0926.

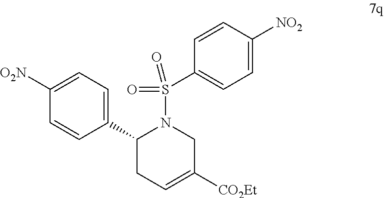 [SMILES: CCOC(=O)C1=CC[C@H](c2ccc([N+](=O)[O-])cc2)N(S(=O)(=O)c2ccc([N+](=O)[O-])cc2)C1]

### Ethyl (R)-6-(4-nitrophenyl)-1-((4-nitrophenyl)sulfonyl)-1,2,5,6-tetrahydropyridine-3-carboxylate (7q)

23.4 mg (52% yield); 96% ee; [α]²⁰_{D} −10.8° (c 1.00, CHCl₃); IR (film) vₘₐₓ 2983, 1709, 1529, 1349, 1267, 1165, 1099; ¹H (500 MHz, CDCl₃) δ 8.36 (d, J=8.9 Hz, 2H), 8.20 (d, J=8.8 Hz, 2H), 8.02 (d, J=8.9 Hz, 2H), 7.49 (d, J=8.5 Hz, 2H), 7.04-7.01 (m, 1H), 5.49 (d, J=6.7 Hz, 1H), 4.49 (d, J=18.8 Hz, 1H), 4.20 (q, J=7.1 Hz, 2H), 3.52-3.47 (m, 1H), 2.78-2.52 (m, 2H), 1.29 (t, J=7.1 Hz, 3H); ¹³C (125 MHz, CDCl₃) δ 163.9, 150.2, 147.7, 145.7, 144.6, 134.8, 128.2, 128.1, 127.7, 124.6, 124.1, 61.3, 52.1, 40.0, 26.9, 14.1; HRMS (ESI) Calculated for [M−H]⁺, C₂₀H₁₈N₃O₈S, m/z 460.0815, found 460.0818.

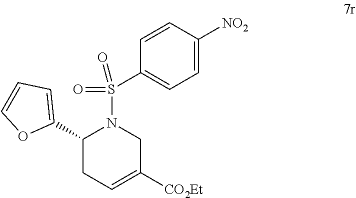 [SMILES: CCOC(=O)C1=CC[C@H](c2ccco2)N(S(=O)(=O)c2ccc([N+](=O)[O-])cc2)C1]

### Ethyl (R)-6-(furan-2-yl)-1-((4-nitrophenyl)sulfonyl)-1,2,5,6-tetrahydropyridine-3-carboxylate (7r)

25.6 mg (63% yield); 93% ee; [α]²⁰_{D} +69.8° (c 1.00, CHCl₃); IR (film) vₘₐₓ 2984, 1709, 1531, 1350, 1267, 1167, 1101; ¹H (500 MHz, CDCl₃) δ 8.30 (d, J=8.9 Hz, 2H), 7.99 (d, J=8.9 Hz, 2H), 7.08 (br d, J=1.2 Hz, 1H), 7.07-7.04 (m, 1H), 6.17 (dd, J=1.8, 3.3 Hz, 1H), 6.02 (d, J=3.3 Hz, 1H), 5.44 (d, J=7.1 Hz, 1H), 4.49 (d, J=17.2 Hz, 1H), 4.22 (q, J=7.2 Hz, 2H), 3.47-3.42 (m, 1H), 2.88-2.82 (m, 1H), 2.68-2.63 (m, 1H), 1.30 (t, J=7.2 Hz, 3H); ¹³C (125 MHz, CDCl₃) δ 164.4, 150.9, 149.9, 145.0, 142.2, 135.3, 128.6, 126.8, 124.0, 110.4, 108.1, 61.1, 47.7, 40.5, 29.3, 14.2; HRMS (ESI) Calculated for [M+H]⁺, C₁₈H₁₉N₂O₇S, m/z 407.0913, found 407.0923.

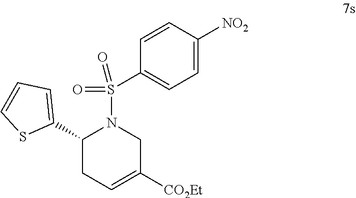 [SMILES: CCOC(=O)C1=CC[C@H](c2cccs2)N(S(=O)(=O)c2ccc([N+](=O)[O-])cc2)C1]

### Ethyl (R)-1-((4-nitrophenyl)sulfonyl)-6-(thiophen-2-yl)-1,2,5,6-tetrahydropyridine-3-carboxylate (7s)

24.8 mg (59% yield); 96% ee; [α]²⁰_{D} +13.6° (c 1.00, CHCl₃); IR (film) vₘₐₓ 2979, 1709, 1531, 1350, 1267, 1167, 1100; ¹H (500 MHz, CDCl₃) δ 8.28 (d, J=8.8 Hz, 2H), 7.94 (d, J=8.8 Hz, 2H), 7.14 (dd, J=1.2, 4.9 Hz, 1H), 7.07-7.05 (m, 1H), 6.86-6.83 (m, 2H), 5.65 (d, J=6.7 Hz, 1H), 4.51 (d, J=17.8 Hz, 1H), 4.22 (q, J=7.2 Hz, 2H), 3.68-3.63 (m, 1H), 2.86-2.82 (m, 1H), 2.70-2.65 (m, 1H), 1.30 (t, J=7.2 Hz, 3H); ¹³C (125 MHz, CDCl₃) δ 164.3, 150.0, 145.2, 140.8, 135.3, 128.4, 127.0, 126.6, 126.3, 125.8, 124.2, 61.1, 49.4, 40.1, 30.9, 14.2; HRMS (ESI) Calculated for [M−H]⁺, C₁₈H₁₇N₂O₆S₂, m/z 421.0528, found 421.0557.

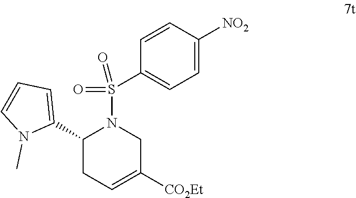 [SMILES: CCOC(=O)C1=CC[C@H](c2cccn2C)N(S(=O)(=O)c2ccc([N+](=O)[O-])cc2)C1]

### Ethyl (R)-6-(1-methyl-1H-pyrrol-2-yl)-1-((4-nitrophenyl)sulfonyl)-1,2,5,6-tetrahydropyridine-3-carboxylate (7t)

30.6 mg (73% yield); 59% ee; [α]²⁰_{D} −22.8° (c 1.00, CHCl₃); IR (film) vₘₐₓ 2977, 1708, 1531, 1349, 1265, 1166, 1098; ¹H NMR (500 MHz, CDCl₃) δ 8.29 (d, J=8.8 Hz, 2H), 7.95 (d, J=8.8 Hz, 2H), 6.81 (t, J=2.4 Hz, 1H), 6.62 (t, J=2.1 Hz, 1H), 5.98 (t, J=3.2 Hz, 1H), 5.90 (dd, J=1.6, 3.3 Hz, 1H), 5.39 (d, J=7.3 Hz, 1H), 4.47 (dd, J=0.8, 18.6 Hz, 1H), 4.18 (q, J=7.1 Hz, 2H), 3.74 (s, 3H), 3.51 (dq, J=3.2, 18.6 Hz, 1H), 2.58 (dq, J=2.8, 19.7 Hz, 1H), 2.41 (dddd, J=2.5, 4.3, 9.7, 19.7 Hz, 1H), 1.28 (t, J=7.1 Hz, 3H); ¹³C NMR (125 MHz, CDCl₃) δ 164.2, 149.9, 145.7, 135.8, 128.1, 127.4, 126.9, 124.1, 123.7, 108.8, 106.7, 60.9, 46.8, 39.7, 34.2, 27.4, 14.1; HRMS (ESI) Calculated for [M+H]⁺, C₁₉H₂₂N₃O₆S, m/z 420.1229, found 420.1209.

### Example II

### Synthesis of R-Aplexone and S-Aplexone

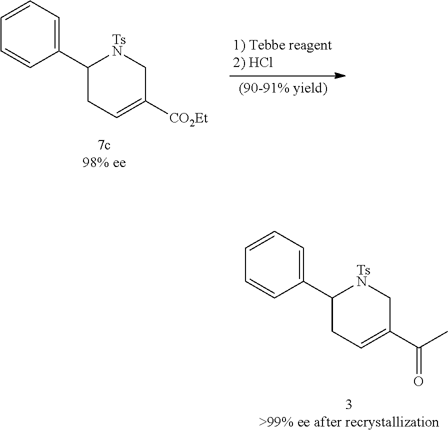 [SMILES: CCOC(=O)C1=CCC(c2ccccc2)N(S(=O)(=O)c2ccc(C)cc2)C1 ; CC(=O)C1=CCC(c2ccccc2)N(S(=O)(=O)c2ccc(C)cc2)C1]

The annulation product (R)-7c was placed in a 10 mL round bottom flask. THF was added (0.2 M), and the reaction was stirred at −40° C. for 10 min. Tebbe reagent (Tebbe reagent prepared as in: Cannizzo, L. F.; Grubbs, R. H. J. Org. Chem. 1985, 50, 2386) in Toluene (˜ 0.7 M; 2 equiv). was added dropwise to the cooled solution. The reaction was allowed to stir for 30 min, then was removed from the cooling bath and stirred for 45 min. The reaction was again cooled to at −40° C. and aqueous NaOH (3 M, 0.5 mL) was added slowly. Upon cessation of gas evolution the reaction was warmed to room temperature and allowed to stir for 1 hr. the reaction was filtered through a plug of silica using EtOAc. The filtrate was concentrated and the residue was dissolved in a HCl solution in acetone (0.1 M) and allowed to stand for 12 hr. The solution was then concentrated and the residue was purified through FCC (SiO₂; EtOAc/Hexane, 15:85) to afforded compound (R)-3 upon concentration to give an oil. The compound can be further purified, with an increase in ee, by recrystallization (EtOAc:Hexane, 2:8).

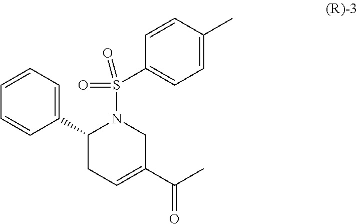 [SMILES: CC(=O)C1=CC[C@H](c2ccccc2)N(S(=O)(=O)c2ccc(C)cc2)C1]

R-Aplexone (R-3) 38.4 mg (90% yield); 99% ee; [α]²⁰_{D} −27.0° (c 0.10, CHCl₃); IR (film) vₘₐₓ 3062, 2920, 1666, 1160 cm⁻¹; ¹H NMR (500 MHz, CDCl₃): δ 7.64 (d, J=8.2 Hz, 2H), 7.27-7.19 (m, 7H), 6.92 (br, 1H), 5.38 (d, J=5.4 Hz, 1H), 4.46 (d, J=18.4 Hz, 1H), 3.38 (ddd, J=18.4, 5.6, 3.3 Hz, 1H), 2.70-2.67 (m, 2H), 2.38 (s, 3H), 2.22 (s, 3H); ¹³C NMR (125 MHz, CDCl₃): δ 196.4, 143.3, 138.2, 137.1, 136.9, 136.2, 129.5, 128.5, 127.7, 127.0, 126.9, 52.1, 38.9, 27.6, 24.9, 21.4; HRMS (m/z): [M+H]⁺ calcd. for C₂₀H₂₁NO₃SH, 356.1315; found, 356.1299.

(S)-Aplexone was synthesized similarly as above using (S)-7c.

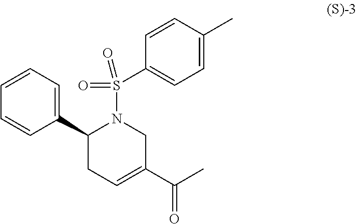 [SMILES: CC(=O)C1=CC[C@@H](c2ccccc2)N(S(=O)(=O)c2ccc(C)cc2)C1]

S-Aplexone (S-3) 38.8 mg (91% yield); 99% ee; [α]²⁰_{D} +27.0° (c 0.10, CHCl₃). The NMR spectral data are identical to that of the R-enantiomer, (R)-3 above.

### Example III

### Aplexone Zebrafish Studies

### Disruption of the Caudal Vein Plexus in Zebrafish Embryos.

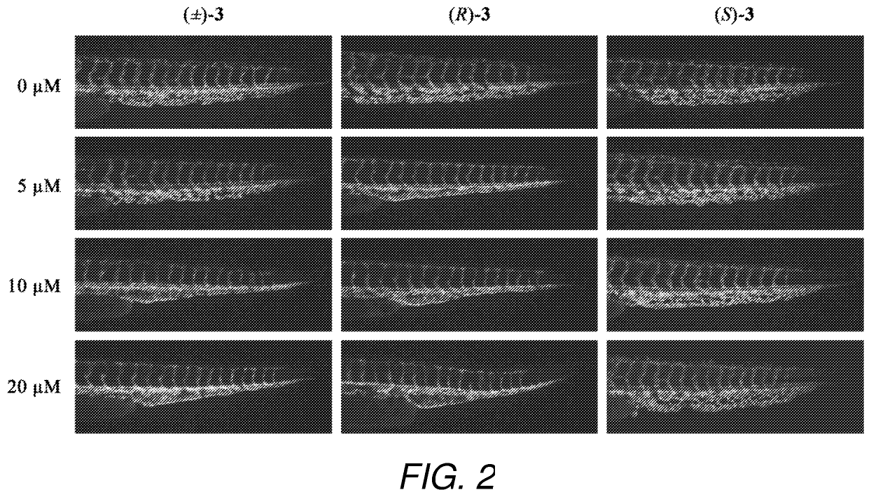

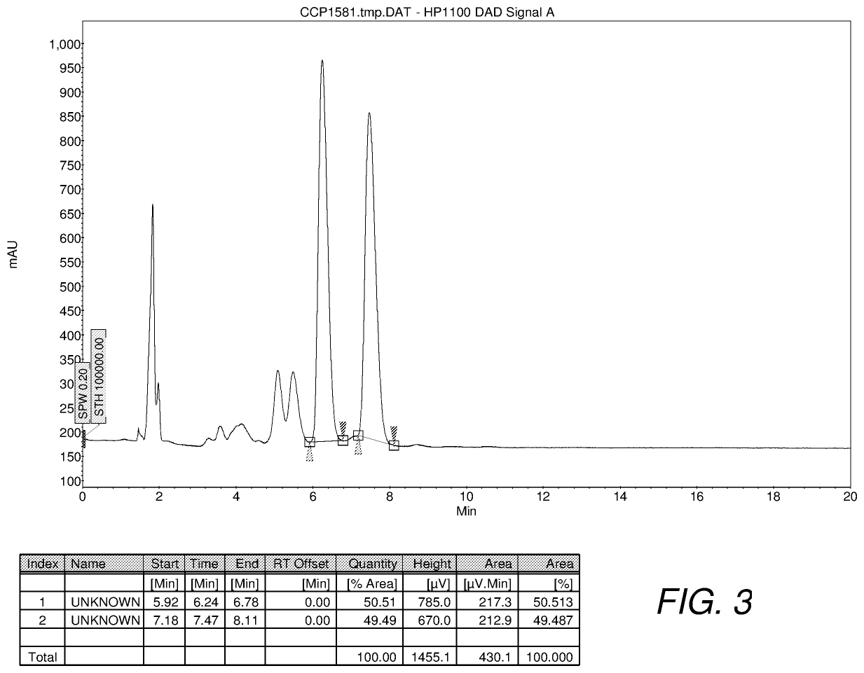

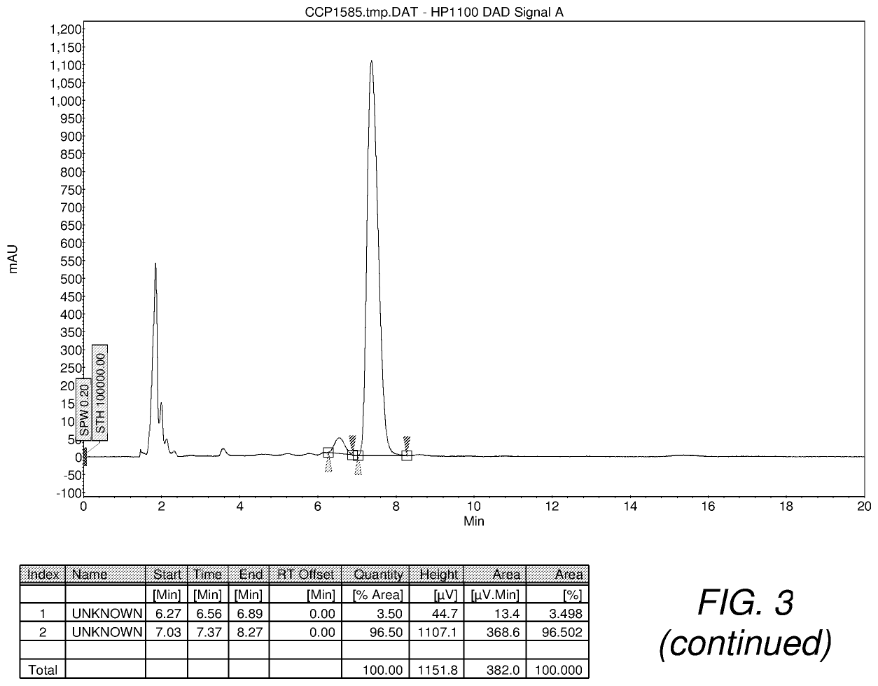

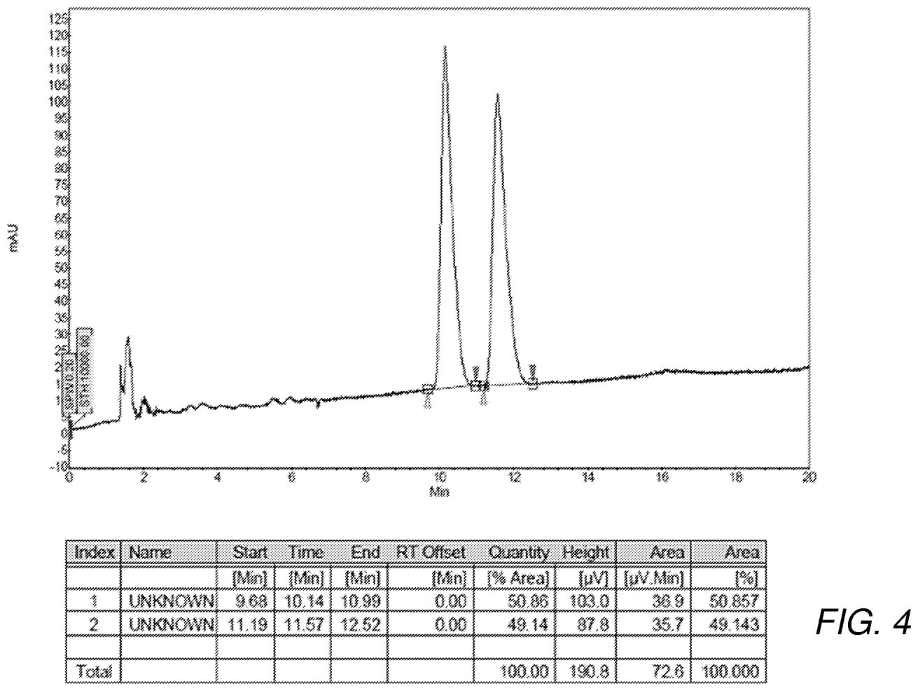

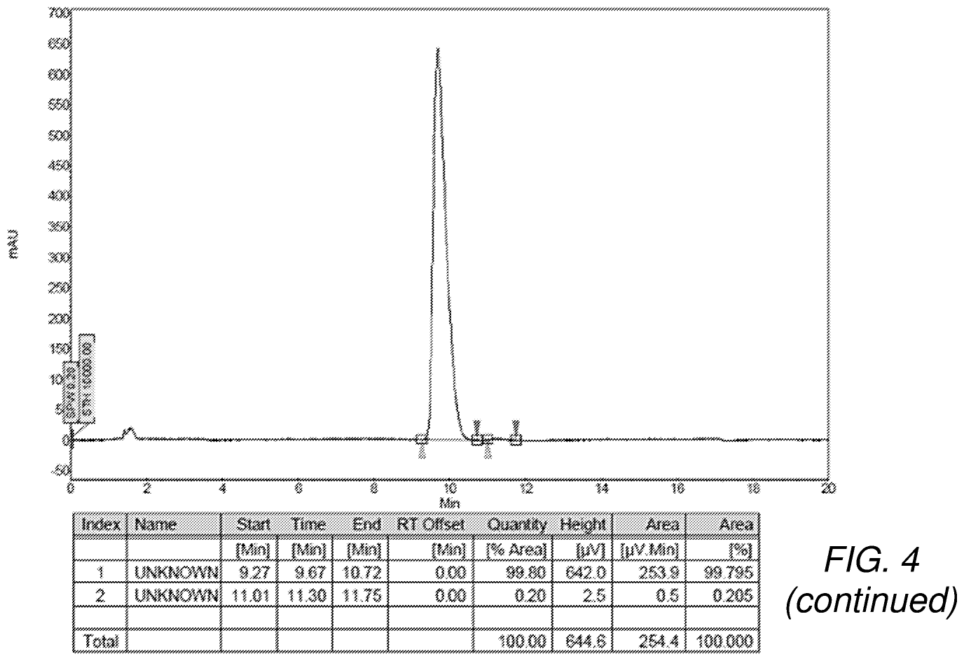

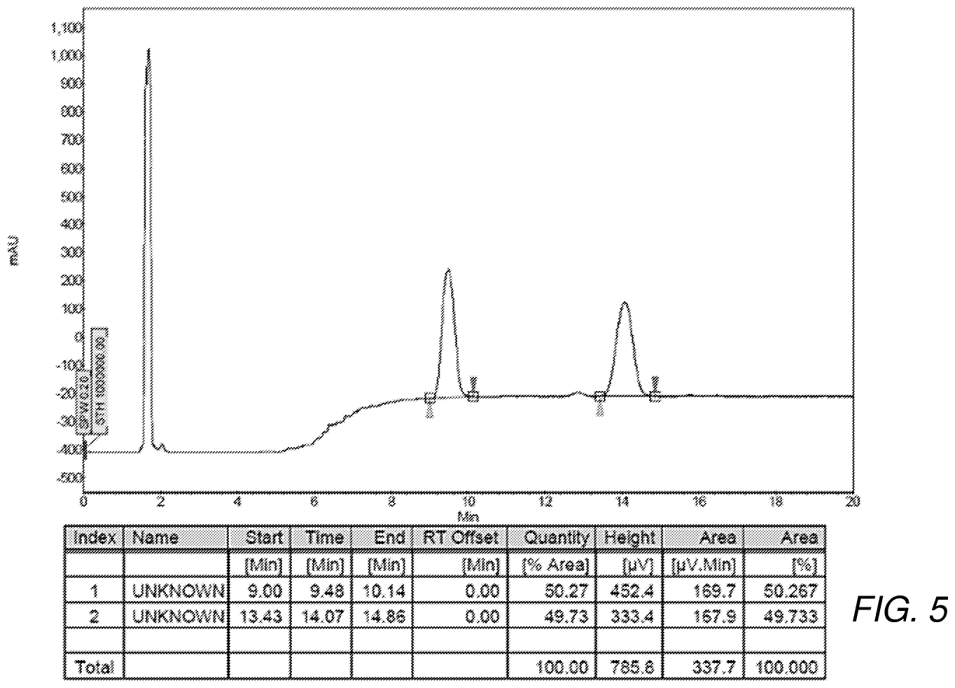

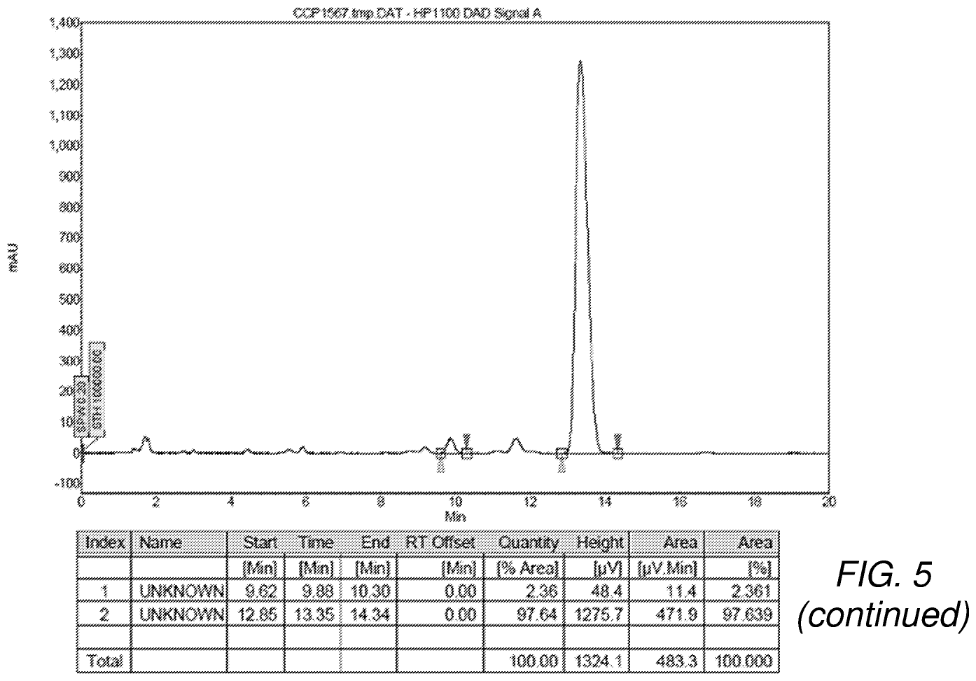

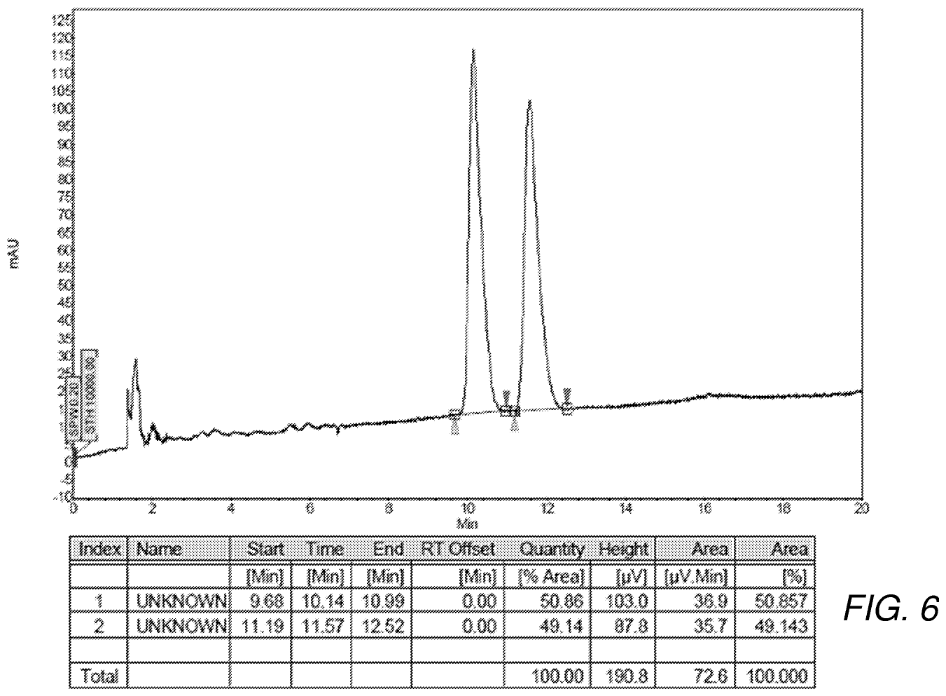

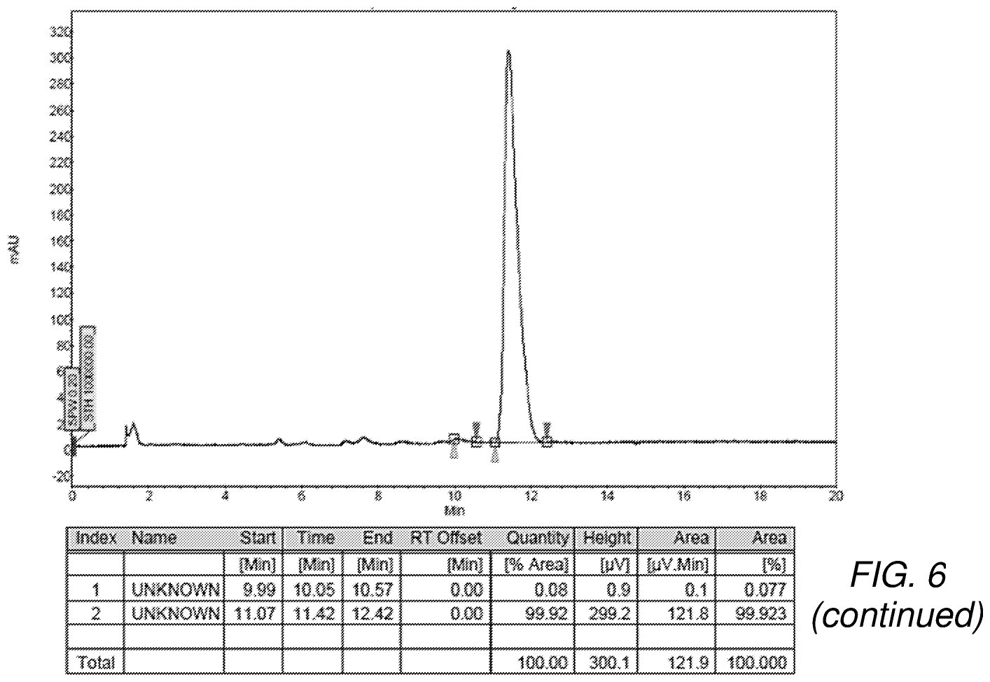

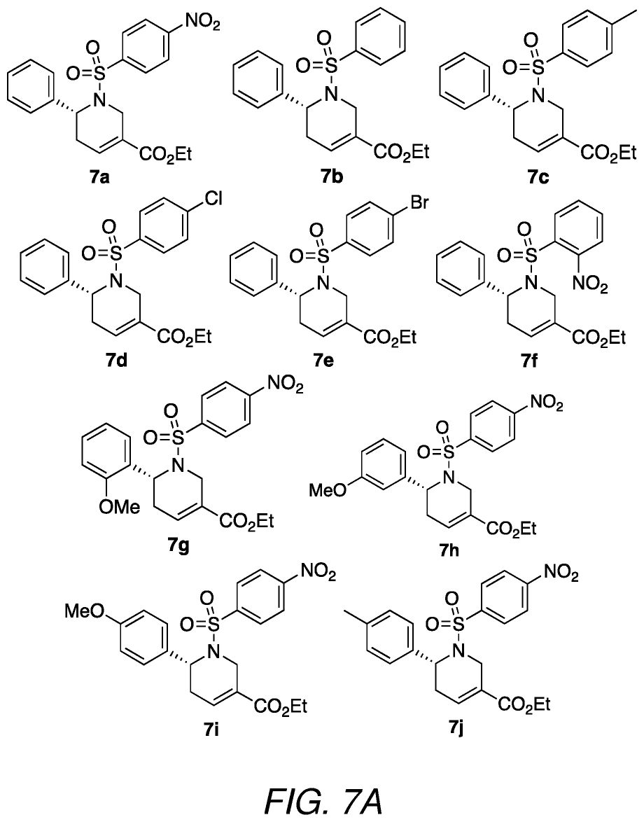

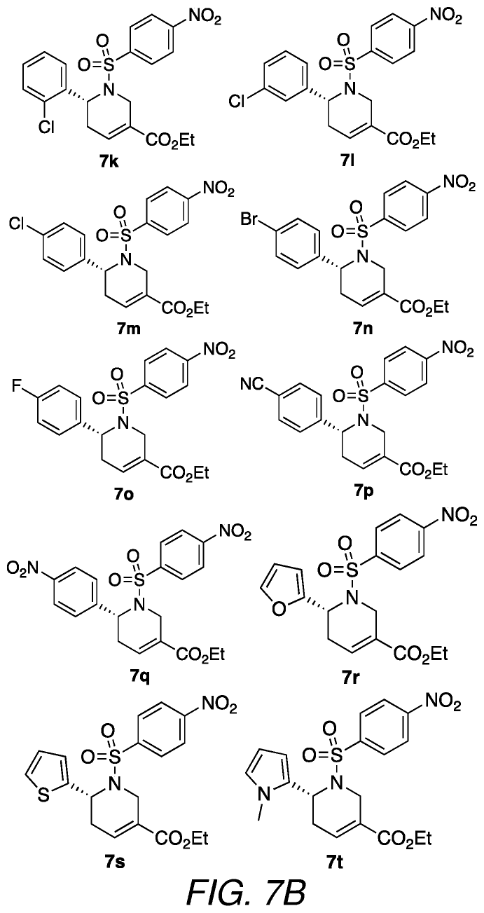

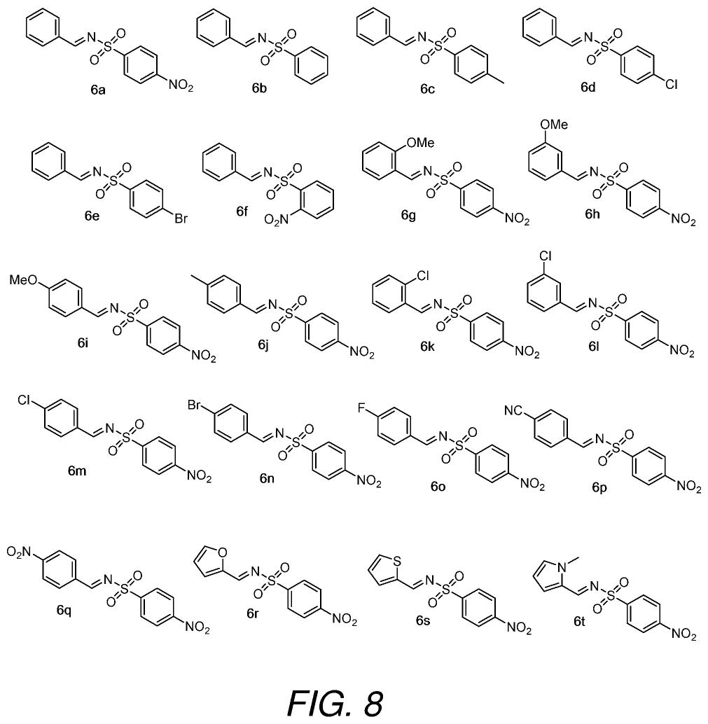

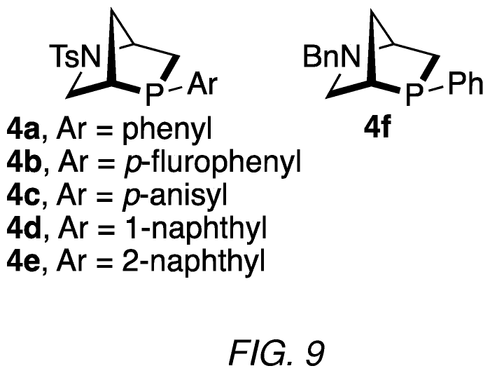

Transgenic Tg(kdrl:GFP)la116 zebrafish embryos were treated with aplexone compounds beginning at 10 hours post fertilization (hpf). After two days of development, treated embryos were anesthetized with 0.01% tricaine and imaged using a Zeiss Axioplan 2 microscope. FIG. 2 shows caudal vein regions of two-day-old zebrafish embryos treated with various concentrations of aplexone. Formation of the caudal vein plexus is disrupted by treatment with 10 μM or higher of racemic (±)-aplexone (3) and 5 μM or higher of R-aplexone (3), but the caudal vein plexus forms normally even after treatment with 20 μM (S)-aplexone (3).

### Cholesterol Assay

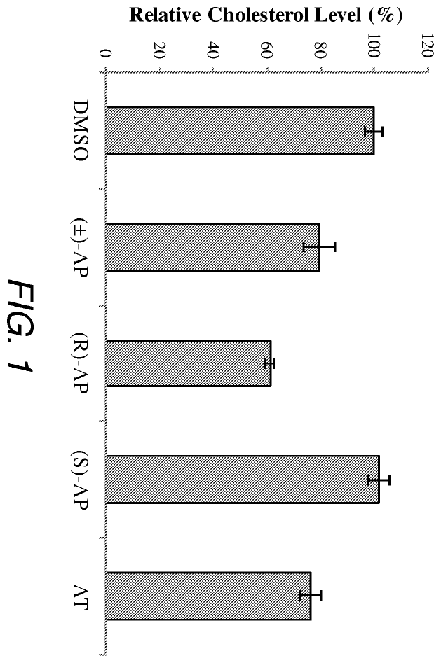

Zebrafish embryos used in cholesterol assays were treated with aplexone or atorvastatin (AT) from 10 hpf to 30 hpf at a concentration of 40 μM. Yolks were removed by vortexing in calcium-free Ringer's solution with 1 mM EDTA at 4° C. The embryos were then washed with calcium-free Ringer's solution, resuspended in cholesterol assay reaction buffer, and lysed by sonication. Cholesterol levels in embryo lysates were measured with an Amplex Red Cholesterol Assay Kit (ThermoFisher Scientific) and FLUOstar Omega microplate reader (BMG Labtech). Protein concentrations of the embryo lysates were measured by DC protein assay (Bio-Rad Laboratories) using a FLUOstar Omega microplate reader. Racemic (±)-Aplexone lowered the cholesterol levels to an extent similar to that of AT. (R)-decreased the cholesterol levels to a greater extent than AT; S-aplexone showed no cholesterol lowering activity (FIG. 1). These results show R-aplexone is the active enantiomer.

1. A composition comprising aplexone, aplexone having a structure of Formula I or a pharmaceutically acceptable salt or solvate thereof, the composition being substantially free of S-stereoisomer of aplexone (S-aplexone)

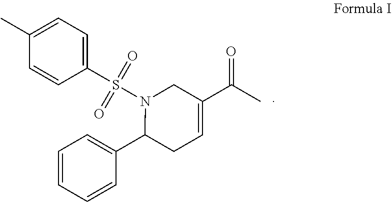 [SMILES: CC(=O)C1=CCC(c2ccccc2)N(S(=O)(=O)c2ccc(C)cc2)C1]

2. The composition of claim 1, wherein the ratio of R-stereoisomer (R-aplexone) to S-aplexone in the composition is greater than 90:10 by weight.

3.-5. (canceled)

6. The composition of claim 1, further comprising a pharmaceutically acceptable carrier.

7. The composition of claim 1, further comprising another agent for cancer treatment and/or another agent for lowering cellular cholesterol levels.

8. A method for treating a subject, comprising administering to the subject a therapeutically effective amount of a composition of claim 1.

9. The method of claim 8, wherein the composition is greater than 10% more effective at treating the subject as compared to the same amount by weight of a mixture of aplexone wherein R-aplexone is not in enantiomeric excess.

10.-13. (canceled)

14. The method of claim 8, wherein angiogenesis is inhibited in the subject.

15. The method of claim 8, wherein cellular cholesterol levels are lowered in the subject.

16. The method of claim 8, wherein 3-hydroxy-3-methylglutaryl-coenzyme A reductase (HMGCR) is reduced in the subject.

17. The method of claim 8, wherein an angiogenesis-mediated condition or disease is treated in the subject.

18. The method of claim 17, wherein the angiogenesis-mediated condition or disease is a cancer.

19. The method of claim 17, wherein the angiogenesis-mediated condition or disease is an inflammatory disease.

20. The method of claim 8, where the subject is human.

21. A method of preparing a compound having a general formula of Formula III 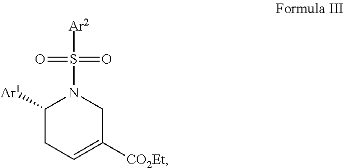
wherein Ar¹ and Ar² are independently an aryl,
the compound having an enantiomeric excess greater than about 80%,
wherein the method comprises the reaction according to the Scheme I

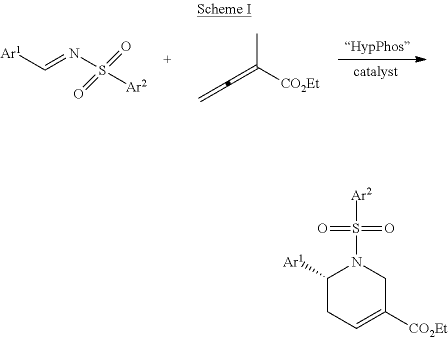

22. The method of claim 21, wherein the HypPhos catalyst is a compound with the general formula of Formula VI 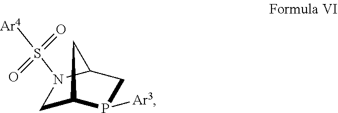
wherein Ar³ is phenyl, p-flurophenyl, p-anisyl, 1-naphthyl or 2-naphthyl and Ar⁴ is aryl, such as p-MeC₆H₄ and the configuration at the phosphorus stereocenter is R.

23. The method of claim 21, wherein Ar² is phenyl, p-nitroC₆H₄, p-MeC₆H₄, p-ClC₆H₄, p-BrC₆H₄, or o-nitroC₆H₄, and Ar¹ is phenyl, o-MeOC₆H₄, m-MeOC₆H₄, p-MeOC₆H₄, p-MeC₆H₄, o-ClC₆H₄, m-ClC₆H₄, p-ClC₆H₄, p-BrC₆H₄, p-FC₆H₄, p-NCC₆H₄, p-NCC₆H₄, 2-furyl, 2-thienyl or 2-N-methylpyrrolyl.

24. The method of claim 21, wherein the reaction is done at ambient temperature and pressure.

25. The method of claim 21, wherein the enantiomeric excess is greater than 90%.

26.-27. (canceled)

28. The method of claim 21, wherein the ratio R-sterioisomer to S-sterioisomer of the compound of formula III obtained from the reaction is greater than 90:10 by weight.

29. The method of claim 21, wherein the compound of general Formula III has the structure of Formula VII [chemical structure]
